(19) United States
(12) Patent Application Publication
(10) Pub. No.: US 2003/0130635 A1
(43) Pub. Date: Jul. 10, 2003

(54) Absorbent articles with compositions having even distribution

(76) Inventors: Martha Lillian Tate, Atlanta, GA (US); Duane Gerard Krzysik, Appleton, WI (US); Hristo Angelov Hristov, Roswell, GA (US); Samuel Qcheng Lin, Paramus, NJ (US)

Correspondence Address: KIMBERLY-CLARK WORLDWIDE, INC., 401 NORTH LAKE STREET, NEENAH, WI, 54956

(21) Appl. No.: 10/028,027

(22) Filed: Dec. 22, 2001

(51) Int. Cl. A61F 13/15

(52) U.S. Cl. 604/367; 604/385.01

(57) ABSTRACT

The present invention relates to absorbent articles including compositions having improved rheological properties. The compositions of the invention exhibit improved spreading characteristics and would provide a more even distribution of skin care compounds on the skin of the wearer of the absorbent articles. The improved spreading characteristics are quantified by a Tangent Delta measurement. The present invention also relates to absorbent articles including compositions that contain silicone elastomers.

### BACKGROUND OF THE INVENTION

The present invention relates to the use of compositions having superior spreading and distribution properties on the body-facing materials of disposable absorbent articles, such as diapers, training pants, adult incontinence products, underpants, feminine care products, nursing pads, wound dressings and similar articles having absorbent capacity. The superior spreading and distribution properties are measured by a Tan Delta rheological characterization. The present invention also relates to the novel use of silicone elastomers in compositions applied to disposable absorbent articles in order to achieve the superior rheology properties.

Absorbent articles such as diapers, training pants, incontinence products and feminine care products are worn such that they are in direct contact with the skin of the wearer. An unavoidable consequence of the use of absorbent articles is that the skin is exposed more directly to various physical and biological insults. Consequently, the barrier function of the skin covered by the absorbent article is put at risk. In order to provide disposability, absorbent articles are primarily constructed of nonwoven materials. Even though nonwoven materials are engineered to have soft hand and drape, they rub against the skin and there is friction. Such friction constitutes one form of physical insult to the skin barrier. Friction against the skin barrier also occurs with the use of absorbent tissues and wipes. Absorbent tissue and wipe products are frequently used for cleansing the skin areas covered by absorbent articles. Absorbent tissue and wipe products are necessary for removing biological waste materials from the skin.

In addition to these physical insults, skin covered by absorbent articles is also frequently exposed to biological insults. Biological fluids, such as urine, feces, vaginal secretions and nasal secretions, may contain a variety of components that can damage the skin barrier. Examples of these components include proteases, lipases and bile acids. Once the skin barrier is compromised, these components, in addition to other constituents of biological fluids, can initiate or exacerbate inflammation of the skin.

Disposable absorbent articles such as diapers, training pants, adult incontinence products, absorbent under pants, feminine care products and nursing pads have been used to absorb body fluids and leave the skin dry. Disposable absorbent articles of this type generally include a liquid impermeable backsheet member, an absorbent core or assembly, and a liquid permeable body facing or liner material. The body facing or liner material comes into contact with the wearer's skin. While the body facing material is made of a soft, compliant material, the material rubs against the skin during use and may not leave the skin completely dry and free of the bodily fluids, such as solid or semi-solid waste, the absorbent article is trying to absorb. During frequent insults of bodily fluids and frequent use of disposable absorbent articles, the skin can become irritated and appear red and be sore to the touch.

Substantial efforts have been made to provide skin care compositions on the bodyfacing surfaces of disposable absorbent articles. The efforts have focused on providing skin care compositions on the bodyside liners of such articles because the bodyside liner typically has the greatest surface area coming into contact with the skin of the wearer of the article. Frequently, the benefits perceived to be obtained from the skin care compositions are only realized if the skin care composition is transferred to the skin of the wearer of the article. Hence, the desire to apply the compositions to the portion of the article having the greatest area of contact with the skin. It is also known to provide skin care compositions on the containment flaps, leg elastics, waist elastics and other portions of absorbent articles that come into direct contact with the wearer's skin.

Description of skin care compositions in the art has shown that both the “chemical” or “formula” make-up of the compositions as well as the physical properties of the compositions are important to the usefulness and efficacy of the compositions for protecting and treating the skin. Chemical properties can include the ability of the composition to act as a barrier against urine, feces or other body exudates or irritants, the ability to maintain or enhance skin barrier function and the ability to breathe and minimize skin overhydration. U.S. Pat. No. 6,153,209 issued to Vega et al. on Nov. 28, 2000 describes the desirability of providing compositions on absorbent articles that are breathable and that act as barriers to irritants.

The physical properties of compositions applied to absorbent articles have also been considered. The melting point, viscosity and hardness of the compositions have been recognized as important physical properties. Skin care compositions having a melting point at or above body temperature can be practically applied to the liner of an absorbent article. In order to be stable on the liner prior to use, the compositions need to be solid at room temperature and at temperatures that may be experienced during transportation and storage. If the compositions melt at room temperature, they will either come off of the liner material or they will soak through the liner and into the absorbent article. For purposes of handling and applying the composition to a liner material, a melting point below the processing temperature is desired. Melting point is managed in the context of formulation needs and other important physical properties like viscosity and hardness.

The viscosity of a skin care composition is important for processing (application to the liner material), stability (on the liner material prior to use) and transfer to the skin from the absorbent article. In some cases, it is desirable for the skin care composition to have a lower viscosity under high shear conditions (such as may be experienced during processing) and a higher viscosity under low shear conditions (such as exist in use on the liner material). In some cases, it is desirable for the composition to be viscoelastic: under low/no shear stress, the composition has appearance of a solid, but as shear stress is increased, the composition can flow as a fluid. Therefore, much work has been done to identify ingredients for the skin care compositions that will deliver a composition having the desired viscosity properties.

Other rheological properties have also been recognized as potentially important to the stability performance of a skin care composition on a liner material. For example, some compositions have particulate ingredients that desirably need to be kept in suspension and prevented from separating or settling out. Additional ingredients have been identified for keeping those particulates suspended, and therefore, maintaining the stability of the composition. One approach to measuring this type of stability is to look at the elastic modulus or apparent viscosity of the composition. PCT publication WO 00/71177 published on Nov. 30, 2000 speculates that elastic modulus relates to the stability of a composition and that apparent viscosity relates to the flowability of the composition. The publication hypothesizes that elastic modulus is a measure of the incomplete dissipation of the energy put into the composition during mixing; residual stored energy is believed to help keep particulates suspended, and therefore, the composition stable. Consequently, formulation directed toward increasing the elastic modulus is believed to be desirable.

In addition to the prior consideration given to some of chemical and physical properties of skin care compositions, the quantity of skin care composition applied to the liner material and the extent of transfer of the composition to the wearer's skin have been considered. Application quantities necessary to overcome migration and other losses have been described as have compositions that transfer in higher amounts to the skin of the wearer of the absorbent article.

Despite the extent of work conducted in relation to skin care compositions being provided on the bodyfacing surfaces of absorbent articles, no consideration has been given to the ability of a skin care composition to transfer from a nonwoven material and to distribute and spread evenly over the skin surface. On a macroscopic level, skin appears to be relatively smooth and consistent; however, on a microscopic level, skin is uneven. The topography of skin includes hills, valleys, discontinuities and other features that disrupt uniformity. The more uniformly a skin care composition can contact the skin surface, the better able the skin care composition is to perform its intended function.

Thus, what is needed is a skin care composition delivered from a bodyside or bodyfacing material of an absorbent article that is able to spread and distribute evenly over the skin surface of a wearer of the absorbent article. It would also be desirable to identify compounds suitable for use in such compositions that are capable of delivering such physical property benefits. Additionally, it would be desirable for the compounds providing the improvements in distribution and spreadability to provide other functions known to be desirable in skin care compositions. Other functions include the ability to reduce irritation of the skin caused by exudates being contained by the absorbent articles and the ability to be “breathable” so as not to block the natural respiration of the skin.

### SUMMARY OF THE INVENTION

In response to the difficulties and problems discussed above, compositions for use on absorbent articles that have improved spreading and distribution characteristics have been discovered. While the compositions of the invention can have a variety of applications, the compositions are particularly beneficial when used in conjunction with absorbent articles such as diapers, incontinence garments, feminine care products, training pants, diaper pants, nursing pads and wound dressings. Additionally, the compositions of the invention can include silicone elastomer compounds. Such compositions exhibit breathability and barrier properties. The purposes and advantages of the present invention will be set forth in and apparent from the description that follows, as well as will be learned by practice of the invention. Additional advantages of the invention will be realized and attained by the compositions and articles particularly pointed out in the written description and claims hereof, as well as from the appended drawings.

In one aspect, the present invention relates to an absorbent article that includes an outer cover, a bodyside liner, an absorbent body and a composition. The bodyside liner is typically liquid permeable and defines a bodyfacing surface. The bodyside liner is connected in a generally superposed relation to the outer cover. The absorbent body is located between the bodyside liner and the outer cover. The composition is on a portion or the entire bodyfacing surface of the bodyside liner. The composition can be generally solid, semi-solid or liquid. The composition may be in a variety of forms, including, but not limited to, emulsions, lotions, creams, ointments, salves, suspensions, gels and the like. The composition can be applied to the bodyside liner using a variety of techniques including foam application, spraying, slot coating and printing. The present invention also encompasses technology that would permit integration of the composition directly with fibers or other materials used to form the bodyside liner. The compositions can be applied to the bodyfacing surface in amounts of from about 0.1 grams per meter squared (g/m²) to about 30 g/m². The compositions of the invention could also be applied to or be present on other skin contacting surfaces of absorbent articles such as the waist and leg elastics and the containment flaps.

The compositions of the invention have improved rheological properties such that the compositions spread and distribute better on the skin of the wearers of the absorbent articles. The ability to spread and distribute better is expressed by the Tangent Delta value of the composition. The Tangent Delta value is an expression of the relationship between the loss modulus of the composition and the elastic shear modulus of the composition. The loss modulus is a measure of the viscous component (ability to spread) of the composition and the elastic modulus is a measure of the elastic component (ability to provide a stable film) of the composition. The compositions of the invention can have a Tangent Delta value of from about 0.10 to about 0.65 as measured over a temperature range of 35 to 40 degrees Celsius. More specifically, the compositions of the invention can have a Tangent Delta value of from about 0.35 to about 0.55 as measured over a temperature range of 35 to 40 degrees Celsius. The Tangent Delta Measurement Procedure is set forth herein.

The loss modulus (represented by G″) and the elastic modulus (represented by G′) of a composition are measured over a range of temperature. The compositions of the invention can have multiple phases that express their individual physical properties as temperature is increased, even though the compositions are stable and uniform at room and storage temperatures. For example, the softening point is the temperature at which at least one “phase” of the composition begins to melt; the softening point is recognized by a change in the slope of the elastic shear modulus (G′) curve. The compositions of the invention can have a softening temperature of from about 15° C. to about 30° C. Desirably, the softening point of a composition is approximately room temperature (about 20° C.).

Materials and compositions that are considered “hard” to the touch can have an elastic modulus on the order of 10⁷ dynes/cm². Materials and compositions that are considered “soft” to the touch can have an elastic modulus that is less than about 10⁵ dynes/cm². When the elastic modulus of a material approaches 10⁶ dynes/cm², the material begins to feel hard. When all of the phases of a composition are in a molten state, the compositions can have a viscous, but liquid consistency. The elastic modulus of viscous liquids is less than about 10⁴ dynes/cm². At body temperature (the temperature experienced by the compositions in use on absorbent articles), the material is typically not liquid yet, so the elastic modulus is something greater than 10⁴ dynes/cm² (the material is typically very soft but has elasticity). The compositions of the invention can have an elastic modulus of from about 10⁵ dynes/cm² to about 10⁷ dynes/cm² over the range of temperatures typically experienced by absorbent articles during transportation, storage and use.

Another relevant aspect of measuring the elastic modulus (G′) over a range of temperature is the slope of the elastic modulus curve. The elastic modulus curve is a plot of the logarithmic value of the elastic modulus versus temperature. When the slope of the elastic modulus curve is steep, the consequence is that the material or composition will become a liquid at a lower temperature (low elastic modulus). The compositions of the invention can have an elastic modulus with a temperature slope of from about −0.06 to about −0.08.

The compositions of the invention can include from about 40 to about 95 percent by weight of one or more emollients. More specifically, the compositions include from about 70 to about 80 percent by weight of emollient(s). Emollients are skin conditioning ingredients that help to soften, smooth, plasticize, lubricate, moisturize, improve the appearance of, improve the feel of and protect the skin. Suitable emollients include, but are not limited to, petroleum based oils, petrolatum, vegetable oils, hydrogenated vegetable oils, animal oils, hydrogenated animal oils, mineral oils, alkyl dimethicones, alkyl methicones, alkyldimethicone copolyols, phenyl silicones, alkyl trimethylsilanes, dimethicone, lanolin and its derivatives, esters, branched esters, glycerol esters and their derivatives, propylene glycol esters and their derivatives, alkoxylated carboxylic acids, alkoxylated alcohols, fatty alcohols, triglycerides, alkyl hydroxystearates and mixtures of such compounds. Those of skill in the art understand that the emollient(s) may also contribute to the rheology and viscosity of the compositions.

The compositions of the invention may also include from about 0.1 to about 40 percent by weight of one or more compounds acting as viscosity enhancers that increase the meltpoint viscosity of the emollients of the composition. More specifically, the compositions include from about 5 to about 20 percent by weight of one or more viscosity enhancers. Even more specifically, the compositions include from about 10 to about 15 percent by weight of viscosity enhancer(s).

Examples of suitable viscosity enhancers include, but are not limited to, polyolefin resins, lipophilic/oil thickeners, ethylene/vinyl acetate copolymers, quaternary starch compounds, natural clays, synthetic analogs of natural clays, organically modified clays, quaternary modified clays, polyethylene, silica, silica silylate, silica methyl silylate, colloidal silicone dioxide, alkyl hydroxy ethyl cellulose, microcrystalline wax, hexadecyl-cosanyl-hexacosanate, shellac wax, glycol montanate, ozokerite wax, C₂₀-C₄₀ alkyl hydroxystearyl stearate, polyperfluoromethylisopropylether montan wax and mixtures of these compounds.

Additionally, the compositions of the invention may include from about 0.1 to about 20 percent by weight of silicone elastomer. The silicone elastomers contribute to the improved rheology of the compositions, contribute to the compositions being breathable and give the compositions an anti-irritant effect. More particularly, the compositions of the invention include from about 5 to about 15 percent by weight of one or more silicone elastomers. Examples of suitable silicone elastomers include, but are not limited to, crosslinked non-emulsifying siloxane elastomers formed from a divinyl compound reacted with Si—H linkages of a polysiloxane; crosslinked non-emulsifying siloxane elastomers formed from a C₃-C₂₀ alkyl polysiloxane compound reacted with Si—H linkages of a polysiloxane and mixtures of these compounds. Examples of suitable crosslinked non-emulsifying siloxane elastomers include Vinyl Dimethicone/Methicone Crosspolymer, Crosslinked Stearyl Methyl Dimethyl Siloxane Copolymer, Dimethicone/Vinyl Dimethicone Crosspolymer, Dimethicone/Phenyl Vinyl Dimethicone Crosspolymer and mixtures of these compounds. Examples of suitable emulsifying silicone elastomers include vinyl MQ resin/organopolysiloxane crosspolymers where the organo group is a polyglycol, polyglycerol, oligosaccharide, hydroxyl-terminated polyoxyalkylene polyether and their carboxylate esters, and lower alkanol ethers, and mixtures of these compounds.

The compositions of the invention can further include from about 5 to about 60 percent by weight of one or more solidifying agents. More specifically, the compositions include from about 25 to about 50 percent by weight of solidifying agents. A solidifying agent is a material capable of solidifying the composition so that the composition is mostly solid at room temperature and has a penetration hardness of at least 5 mm. More specifically, the solidifying agent includes one or more materials that are capable of solidifying the natural fats/oils and emollient combination so as to have a penetration hardness of 5 to about 365 mm at 25° C. Further, the solidifying agent solidifies the emollient (or the fat/oil/emollient combination when fats and oils are used in the composition) so that it has a melting point between 32° C. and 100° C. Those of skill in the art will recognize that the solidifying agent component or combination of solidifying agents may also contribute to the rheology and viscosity of the compositions.

One or more solidifying agents can be selected from beeswax, behenyl behenate, behenyl benzoate, branched esters, candelilla wax, carnauba wax, synthetic carnauba wax, PEG-12 carnauba wax, cerasin, microcrystalline wax, hydrogenated microcrystalline wax, hexadecylcosanyl hexacosanate, polyperfluoromethylisopropylether montan wax, alkylmethylsiloxanes, glycol montanate, jojoba wax, lanolin wax, ozokerite, paraffin, synthetic paraffin, polyethylene, C₂₀-C₄₀ alkyl hydroxystearyl stearate, C₃₀ alkyl dimethicone, cetyl esters, zinc stearate, shellac wax, hydrogenated cottonseed oil, hydrogenated squalene, hydrogenated jojoba oil and mixtures of such compounds.

The compositions of the invention can also include from about 0.1 to about 59 percent by weight of natural fats or natural oils. More specifically, the compositions can include from about 10 to about 50 percent by weight of natural fats or natural oils. Natural fats and oils include fats, oils, essential oils, fatty acids, fatty alcohols, phospholipids and mixtures of these compounds. The natural fats and oils can be similar to the lipids that are present in healthy skin in order to mimic the naturally present lipids. Synthetic or synthetically modified fats and oils could potentially also be used if they functioned in the same manner as their natural counterparts. Examples of fats and oils include, but are not limited to, Avocado Oil, Apricot Oil, Babassu Oil, Borage Oil, Camellia Oil, Canola Oil, Castor Oil, Coconut Oil, Corn Oil, Cottonseed Oil, Evening Primrose Oil, Hydrogenated Cottonseed Oil, Hydrogenated Palm Kernel Oil, Maleated Soybean Oil, Meadowfoam Oil, Palm Kernel Oil, Peanut Oil, Rapeseed Oil, Safflower Oil, Sphingolipids, Sweet Almond Oil, Tall Oil, Lanolin, Lanolin Alcohol, Lauric Acid, Palmitic Acid, Stearic Acid, Linoleic Acid, Stearyl Alcohol, Lauryl Alcohol, Myristyl Alcohol, Behenyl Alcohol, Rose Hip Oil, Calendula Oil, Chamomile Oil, Eucalyptus Oil, Juniper Oil, Sandlewood Oil, Tea Tree Oil, Sunflower Oil, Soybean Oil and mixtures thereof.

The compositions can also include sterols, sterol derivatives or mixtures of both in an amount of from about 0.1 to about 10 percent by weight. Sterols and sterol derivatives include compounds such as β-sterols with a tail on the 17 position and no polar groups, such as cholesterol, C₁₀-C₃₀ cholesterol/lanosterol esters, tall oil sterols, soy sterols, sterol esters and mixtures of these compounds. More specifically, the compositions include from about 0.5 to about 5 percent by weight of sterols, sterol derivatives or mixtures of both. Even more specifically, the compositions include from about 0.8 to about 1 percent by weight of the sterol compounds. Examples of suitable sterol compounds include, but are not limited to, cholesterol, sitosterol, stigmasterol, and ergosterol, as well as, C₁₀-C₃₀ cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyldecanoate, dihydrolanosterol, dihydrolanosteryl octyldecanoate, ergocalciferol, tall oil sterol, soy sterol acetate, lanasterol, soy sterol, avocado sterols, “AVOCADIN” (available from Croda Ltd. of Parsippany, N.J.), sterol esters and mixtures thereof.

In addition to the components already described, the compositions of the invention may also include active, skin care ingredients such as those ingredients that may be useful for treating skin irritations such as diaper rash. Examples of such active ingredients include allantoin and its derivatives, aloe, aluminum hydroxide gel, calamine, cocoa butter, cod liver oil, dimethicone, glycerin, kaolin and its derivatives, lanolin and its derivatives, mineral oil, petrolatum, white petrolatum, shark liver oil, talc, topical starch, zinc acetate, zinc carbonate, zinc oxide and mixtures of these ingredients. Some of the ingredients listed as possible skin care ingredients for treating the skin can also be used as emollients.

Ranges are used to describe the relative quantities of compounds in the compositions of the invention and ranges are used to describe the relative physical properties of the compositions of the invention. It is understood that the ranges are by way of illustration only and that one of skill in the art would recognize that the nature of the specific compositions dictates the levels to be applied to achieve the desired results. The levels of components are ascertainable by routine experimentation in view of the present disclosure.

The compositions of the invention typically have a melting point of from about 32° C. to about 100° C. Melting behavior in this range provides compositions that are relatively immobile and localized on the bodyfacing surface of the bodyside liner of the absorbent article at room temperature. Though relatively immobile and localized at room temperature, the compositions are also readily transferable to the wearer of the article at body temperature through natural rubbing or friction during wearing and through adhesion of the composition to the skin of the wearer. The compositions also maintain their integrity and are not completely liquid at elevated temperatures such as may be experienced during storage. Stability in a solid state at elevated temperatures is made possible, in part, by the increase in viscosity provided by the viscosity enhancers. Desirably, the compositions of the invention are easily transferable to the skin by way of normal contact, including adhesion of the composition to the skin, wearer motion or body heat. Because the compositions are relatively immobilized on the bodyfacing surfaces of the articles, the quantities of the compositions necessary to provide the desired skin barrier benefits are reduced. In addition, special barrier or wrapping materials may not be necessary for the articles of the invention.

The compositions of the invention have high shear viscosities of less than about 5,000 centipoise at processing temperatures such as at a temperature of about 60° C. or higher. The melting points and, therefore, the processing temperatures vary for different compositions of the invention. At about 55° C. or less, the compositions have low shear viscosities greater than about 50,000 centipoise. The compositions may also have a penetration hardness of from about 5 millimeters to about 365 millimeters at 25° C.

The absorbent articles and compositions of the invention advantageously protect the skin barrier and subdue inflammation and exhibit an improved rheology not observed with conventional absorbent articles and compositions. It is to be understood that both the foregoing general description and the following detailed description are exemplary and are intended to provide further explanation of the invention claimed. The accompanying drawings, that are incorporated in and constitute part of this specification, are included to illustrate and provide a further understanding of the articles and compositions of the invention. Together with the description, the drawings serve to explain the various aspects of the invention.

### BRIEF DESCRIPTION OF THE DRAWINGS

The present invention will be more fully understood and further advantages will become apparent when reference is made to the following detailed description of the invention and the accompanying drawings. The drawings are merely representative and are not intended to limit the scope of the claims. Like parts of the absorbent articles depicted in the drawings are referred to by the same reference numerals.

FIG. 1 representatively shows a partially cut away, top plan view of an absorbent article according to one aspect of the invention in a stretched and laid flat condition with the surface of the article that contacts the skin of the wearer facing the viewer;

FIG. 2 graphically represents the relationship between elastic modulus (G′) and temperature for a composition of the invention; and

FIG. 3 graphically represents the relationship between elastic modulus (G′) and temperature for compositions of the invention and previously known compositions.

### DETAILED DESCRIPTION OF THE INVENTION

The present invention is directed to identifying compositions delivered from a bodyfacing surface of an absorbent article that, once transferred to the skin of the wearer of the article, distribute more evenly on the skin to provide a better barrier to irritants than previously known compositions. The compositions of the invention have balanced spreading and stable film-forming properties. The present invention is also directed to compositions used on absorbent articles that have a silicone elastomer component.

The present disclosure of the invention will be expressed in terms of its various components, elements, constructions, configurations, arrangements and other features that may also be individually or collectively referenced by the term, “aspect(s)” of the invention, or other similar terms. It is contemplated that the various forms of the disclosed invention may incorporate one or more of its various features and aspects, and that such features and aspects may be employed in any desired, operative combination thereof.

It should also be noted that when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components or groups thereof.

The present invention encompasses compositions as they are applied to the bodyfacing materials of absorbent articles and absorbent articles including compositions. The following detailed description will be made in the context of one type of absorbent article, a disposable diaper that is adapted to be worn by infants about their lower torso. It is readily apparent, however, that the absorbent article of the present invention would also be suitable for use as another type of absorbent article, such as a feminine care pad, an incontinence garment, a training pant, a prefastened or refastenable diaper pant, a wound dressing or a nursing pad. Further, the compositions of the invention are not limited to application on the bodyfacing materials of absorbent articles. For example, the compositions of the inventions could be used on skin-contacting substrates such as tissues, wet (pre-moistened) wipe materials and cosmetic pads (such as for cleansing or buffing).

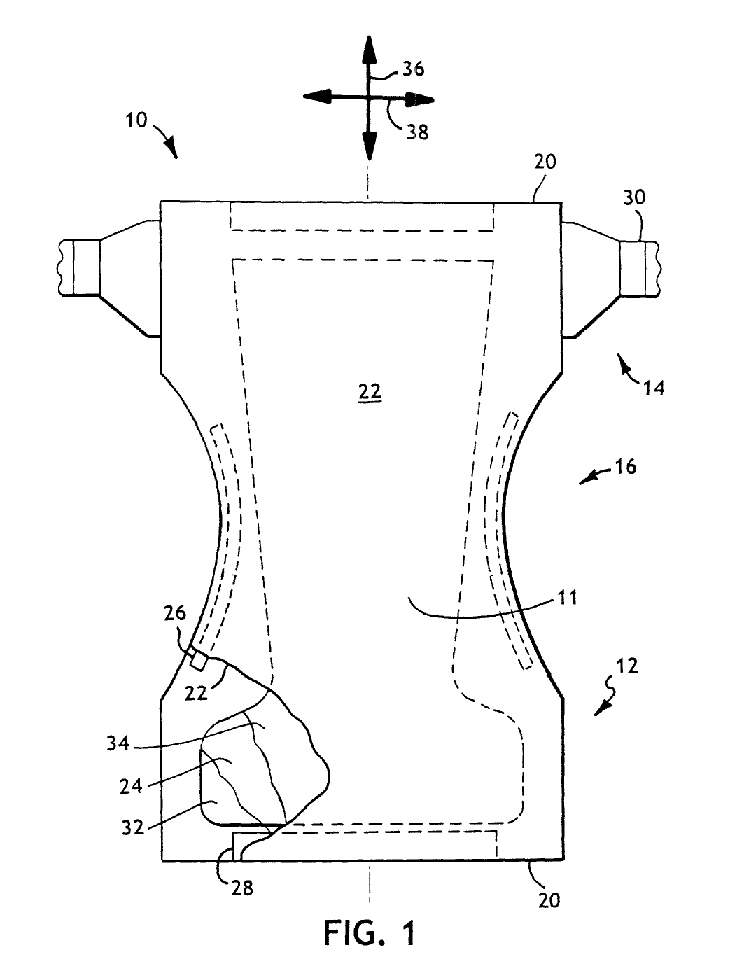

FIG. 1 is a representative plan view of a disposable diaper 10 of the present invention in a flat-out, uncontracted state (i.e., with all elastic induced gathering and contraction removed). The bodyfacing surface 11 of the diaper 10, that is, the surface 11 of the diaper 10 that contacts the wearer is facing the viewer. The compositions of the invention can be applied to one or more bodyfacing materials that are components of the diaper 10. As used herein, the term ‘bodyfacing material’ includes, but is not limited to, materials such as the bodyside liner or topsheet, elastic material, tissue, intake and distribution material, absorbent material, and backsheet material. Each of these materials and components of a diaper 10 are described more fully herein. The compositions of the invention are applied to one or more of the bodyfacing materials in order to have a beneficial protective effect on the skin barrier. The bodyfacing material of the present invention can be a single layer or multi-layered.

With reference to FIG. 1, the diaper 10 generally defines a front waist section 12, a rear waist section 14, and an intermediate section 16 that interconnects the front and rear waist sections 12 and 14. The front and rear waist sections 12 and 14 include the general portions of the diaper 10 that are constructed to extend substantially over the wearer's front and rear abdominal regions, respectively, during use. The intermediate section 16 of the diaper 10 includes the general portion of the diaper 10 that is constructed to extend through the wearer's crotch region between the legs.

The diaper 10 includes a vapor permeable backsheet or outer cover 20, a liquid permeable topsheet or bodyside liner 22 positioned in facing relation with the outer cover 20, and an absorbent body 24, such as an absorbent pad, which is located between the outer cover 20 and the bodyside liner 22. The outer cover 20 defines a length and a width that, in the illustrated aspect, coincide with the length and width of the diaper 10. The absorbent body 24 generally defines a length and width that are less than the length and width of the outer cover 20, respectively. Thus, marginal portions of the diaper 10, such as marginal sections of the outer cover 20, may extend past the terminal edges of the absorbent body 24. In the illustrated aspects, for example, the outer cover 20 extends outwardly beyond the terminal marginal edges of the absorbent body 24 to form side margins and end margins of the diaper 10. The bodyside liner 22 is generally coextensive with the outer cover 20 but may optionally cover an area that is larger or smaller than the area of the outer cover 20, as desired. In other words, the bodyside liner 22 is connected in superposed relation to the outer cover 20. The outer cover 20 and bodyside liner 22 are intended to face the garment and body of the wearer, respectively, while in use.

To provide improved fit and to help reduce leakage of body exudates from the diaper 10, the diaper side margins and end margins may be elasticized with suitable elastic members, such as single or multiple strands of elastic. The elastic strands may be composed of natural or synthetic rubber and may optionally be heat shrinkable or heat elasticizable. For example, as representatively illustrated in FIG. 1, the diaper 10 may include leg elastics 26 which are constructed to operably gather and shirr the side margins of the diaper 10 to provide elasticized leg bands which can closely fit around the legs of the wearer to reduce leakage and provide improved comfort and appearance. Similarly, waist elastics 28 can be employed to elasticize the end margins of the diaper 10 to provide elasticized waists. The waist elastics 28 are configured to operably gather and shirr the waist sections to provide a resilient, comfortably close fit around the waist of the wearer. In the illustrated aspects, the elastic members are illustrated in their uncontracted, stretched condition for the purpose of clarity.

Fastening means, such as hook and loop fasteners 30, are employed to secure the diaper 10 on a wearer. Alternatively, other fastening means, such as buttons, pins, snaps, adhesive tape fasteners, cohesives, mushroom-and-loop fasteners, or the like, may be employed. Additionally, more than two fasteners can be provided, particularly if the diaper 10 is to be provided in a prefastened configuration. The fasteners can vary in size and form.

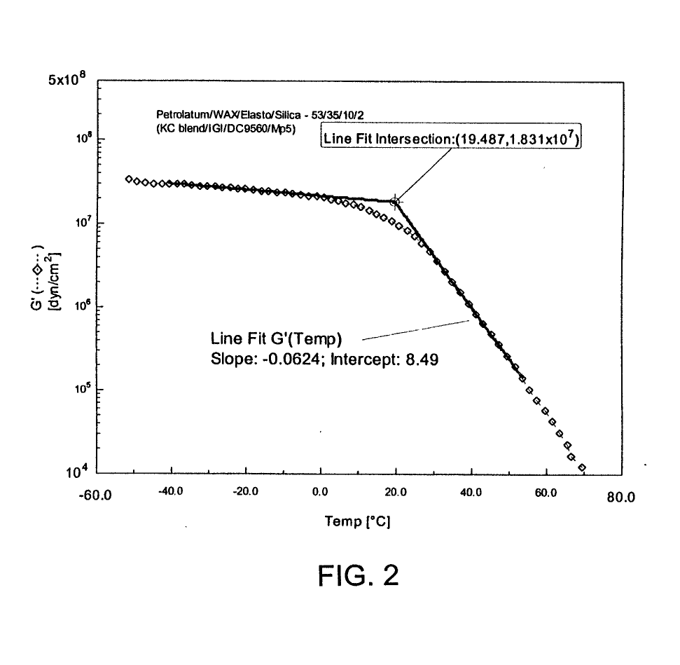

The diaper 10 may further include other layers between the absorbent body 24 and the bodyside liner 22 or outer cover 20. For example, as representatively illustrated in FIGS. 1 and 2, the diaper 10 may include a ventilation layer 32 located between the absorbent body 24 and the outer cover 20 to insulate the outer cover 20 from the absorbent body 24, to improve air circulation and to effectively reduce the dampness of the garment facing surface of the outer cover 20. The ventilation layer 32 may also assist in distributing fluid exudates to portions of the absorbent body 24 that do not directly receive the insult. The diaper 10 may also include a surge management layer 34 located between the bodyside liner 22 and the absorbent body 24 to prevent pooling of the fluid exudates and further improve air exchange and distribution of the fluid exudates within the diaper 10.

The diaper 10 may be of various suitable shapes. For example, the diaper may have an overall rectangular shape, T-shape or an approximately hourglass shape. In the shown aspect, the diaper 10 has a generally I-shape. The diaper 10 further defines a longitudinal direction 36 and a lateral direction 38. Other suitable diaper components that may be incorporated on absorbent articles of the present invention include containment flaps, waist flaps, elastomeric side panels, and the like which are generally known to those skilled in the art. Likewise, if the diaper 10 is to be sold in a prefastened condition, the diaper 10 may have passive bonds (not shown) that join the rear waist section 14 with the front waist section 12.

Examples of diaper configurations suitable for use in connection with the instant application that may include other diaper components suitable for use on diapers are described in U.S. Pat. No. 4,798,603 issued Jan. 17, 1989, to Meyer et al.; U.S. Pat. No. 5,176,668 issued Jan. 5, 1993, to Bernardin; U.S. Pat. No. 5,176,672 issued Jan. 5, 1993, to Bruemmeret al.; U.S. Pat. No. 5,192,606 issued Mar. 9, 1993, to Proxmire et al.; U.S. Pat. No. 5,496,298 issued Mar. 5, 1996 to Kuepper et al.; and U.S. Pat. No. 5,509,915 issued Apr. 23, 1996 to Hanson et al., the disclosures of which are herein incorporated by reference.

The various components of the diaper 10 are integrally assembled together employing various types of suitable attachment means, such as adhesive, sonic bonds, thermal bonds or combinations thereof. In the shown aspect, for example, the bodyside liner 22 and outer cover 20 are assembled to each other and to the absorbent body 24 with lines of adhesive, such as a hot melt, pressure-sensitive adhesive. Similarly, other diaper components, such as the elastic members 26 and 28, fastening members 30, and ventilation and surge layers 32 and 34 may be assembled into the diaper 10 by employing the above-identified attachment mechanisms.

The outer cover 20 of the diaper 10, as representatively illustrated in FIG. 1, is composed of a substantially vapor permeable material. The permeability of the outer cover 20 is configured to enhance the breathability of the diaper 10 and to reduce the hydration of the wearer's skin during use without allowing excessive condensation of vapor, such as urine, on the garment facing surface of the outer cover 20 that can undesirably dampen the wearer's clothes. The outer cover 20 is generally constructed to be permeable to at least water vapor and has a water vapor transmission rate of at least about 1000 g/m²/24 hr., desirably at least about 1500 g/m²/24 hr, more desirably at least about 2000 g/m²/24 hr., and even more desirably at least about 3000 g/m²/24 hr. For example, the outer cover 20 may define a water vapor transmission rate of from about 1000 to about 6000 g/m²/24 hr. Materials that have a water vapor transmission rate less than those above do not allow a sufficient amount of air exchange and undesirably result in increased levels of skin hydration.

The outer cover 20 is also desirably substantially liquid impermeable. For example, the outer cover 20 may be constructed to provide a hydrohead value of at least about 60 cm, desirably at least about 80 cm, and more desirably at least about 100 cm when subjected to the Hydrostatic Pressure Test. Materials that have hydrohead values less than those above undesirably result in the strike through of liquids, such as urine, during use. Such fluid strike through can undesirably result in a damp, clammy feeling on the outer cover 20 during use. The methods by which Water Vapor Transmission Rate and Hydrostatic Pressure can be measured are described in U.S. Pat. No. 6,217,890 issued Apr. 17, 2001 to Paul et al. and incorporated herein by reference.

The outer cover 20 may be composed of any suitable materials that either directly provide the above desired levels of liquid impermeability and air permeability or, in the alternative, materials that can be modified or treated in some manner to provide such levels. In one aspect, the outer cover 20 may be a nonwoven fibrous web constructed to provide the required level of liquid impermeability. For example, a nonwoven web composed of spunbond or meltblown polymer fibers may be selectively treated with a water repellent coating or laminated with a liquid impermeable, vapor permeable polymer film to provide the outer cover 20. In a particular aspect of the invention, the outer cover 20 may include a nonwoven web composed of a plurality of randomly deposited hydrophobic thermoplastic meltblown fibers that are sufficiently bonded or otherwise connected to one another to provide a substantially vapor permeable and substantially liquid impermeable web. The outer cover 20 may also include a vapor permeable nonwoven layer that has been partially coated or otherwise configured to provide liquid impermeability in selected areas.

Examples of suitable materials for the outer cover 20 are also described in U.S. Pat. No. 5,482,765 issued Jan. 9, 1996 in the name of Bradley et al. and entitled “NONWOVEN FABRIC LAMINATE WITH ENHANCED BARRIER PROPERTIES”; U.S. Pat. No. 5,879,341 issued Mar. 9, 1999 in the name of Odorzynski et al. and entitled “ABSORBENT ARTICLE HAVING A BREATHABILITY GRADIENT”; U.S. Pat. No. 5,843,056 issued Dec. 1, 1998, in the name of Good et al. and entitled “ABSORBENT ARTICLE HAVING A COMPOSITE BREATHABLE BACKSHEET”; and U.S. Pat. No. 6,309,736 issued Oct. 30, 2001, in the name of McCormack et al. and entitled “LOW GAUGE FILMS AND FILM/NONWOVEN LAMINATES”, the disclosures of which are herein incorporated by reference.

In a particular aspect, the outer cover 20 is provided by a microporous film/nonwoven laminate material that includes a spunbond nonwoven material laminated to a microporous film. The spunbond nonwoven comprises filaments of about 1.8 denier extruded from a copolymer of ethylene with about 3.5 weight percent propylene and defines a basis weight of from about 17 to about 25 grams per square meter. The film includes a cast coextruded film having calcium carbonate particles therein and defines a basis weight of about 58 grams per square meter prior to stretching. The film is preheated, stretched and annealed to form the micropores and then laminated to the spunbond nonwoven. The resulting microporous film/nonwoven laminate based material has a basis weight of from about 30 to about 60 grams per square meter and a water vapor transmission rate of from about 3000 to about 6000 g/m²/24 hr. Examples of such film/nonwoven laminate materials are described in more detail in U.S. Pat. No. 6,309,736 issued Oct. 30, 2001, in the name of McCormack et al. and entitled “LOW GAUGE FILMS AND FILM/NONWOVEN LAMINATES”, the disclosure of which has been incorporated by reference.

In another aspect, the outer cover 20 is provided by an extensible material. Further, the outer cover 20 can also be provided by a material having stretch in both the longitudinal 36 and lateral 38 directions. Extensible and stretchable outer cover materials can be used in absorbent articles to provide various benefits including better fitting articles.

The bodyside liner 22, as representatively illustrated in FIG. 1, defines a bodyfacing surface 11 that is compliant, soft feeling, and nonirritating to the wearer's skin. Further, the bodyside liner 22 may be less hydrophilic than the absorbent body 24, to present a relatively dry surface to the wearer, and may be sufficiently porous to be liquid permeable, permitting liquid to readily penetrate through its thickness. A suitable bodyside liner 22 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The bodyside liner 22 is suitably employed to help isolate the wearer's skin from liquids held in the absorbent body 24.

Various woven and nonwoven fabrics can be used for the bodyside liner 22. For example, the bodyside liner 22 may be composed of a meltblown or spunbond web of polyolefin fibers. The bodyside liner 22 may also be a bonded-carded web composed of natural and/or synthetic fibers. The bodyside liner 22 may be composed of a substantially hydrophobic material, and the hydrophobic material may, optionally, be treated with a surfactant or otherwise processed to impart a desired level of wettability and hydrophilicity. In a particular aspect of the present invention, the bodyside liner 22 includes a nonwoven, spunbond, polypropylene fabric composed of about 2.8-3.2 denier fibers formed into a web having a basis weight of about 22 grams per square meter and a density of about 0.06 gram per cubic centimeter.

In a particular aspect of the present invention, the bodyside liner 22 may be surface treated with about 0.3 weight percent of a surfactant mixture that contains a mixture of AHCOVEL Base N-62 and GLUCOPON 220UP surfactants in about a 3:1 ratio based on a total weight of the surfactant mixture. The AHCOVEL Base N-62 surfactant is purchased from Hodgson Textile Chemicals Inc., a business having offices in Mount Holly, N.C., and includes a blend of hydrogenated ethoxylated castor oil and sorbitan monooleate in a 55:45 weight ratio. The GLUCOPON 220UP surfactant is purchased from Henkel Corporation and includes alkyl polyglycoside. The surfactant may also include additional ingredients such as aloe. The surfactant may be applied by any conventional means, such as spraying, printing, brush coating, foam or the like. The surfactant may be applied to the entire bodyside liner 22 or may be selectively applied to particular sections of the bodyside liner 22, such as the medial section along the longitudinal centerline of the diaper, to provide greater wettability of such sections.

The absorbent body 24 of the diaper 10, as representatively illustrated in FIG. 1, may suitably comprise a matrix of hydrophilic fibers, such as a web of cellulosic fluff, mixed with particles of a high-absorbency material commonly known as superabsorbent material. In a particular aspect, the absorbent body 24 includes a matrix of cellulosic fluff, such as wood pulp fluff, and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be nonuniformly mixed. Alternatively, the absorbent body 24 may include a laminate of fibrous webs and superabsorbent material or other suitable matrix for maintaining a superabsorbent material in a localized area.

The absorbent body 24 may have any of a number of shapes. For example, the absorbent body 24 may be rectangular, I-shaped, or T-shaped. It is generally preferred that the absorbent body 24 is narrower in the intermediate section than in the front or rear waist sections of the diaper 10. The absorbent body 24 may be provided by a single layer or, in the alternative, may be provided by multiple layers, all of which need not extend the entire length and width of the absorbent body 24. In a particular aspect of the invention, the absorbent body 24 can be generally T-shaped with the laterally extending cross-bar of the “T” generally corresponding to the front waist section 12 of the absorbent article for improved performance, especially for male infants. In the illustrated aspects, for example, the absorbent body 24 across the front waist section 12 of the article has a cross-directional width of about 18 centimeters, the narrowest portion of the intermediate section 16 has a width of about 7.5 centimeters and in the rear waist section 14 has a width of about 11.4 centimeters.

The size and the absorbent capacity of absorbent body 24 should be compatible with the size of the intended wearer and the liquid loading imparted by the intended use of the absorbent article. Further, the size and the absorbent capacity of the absorbent body 24 can be varied to accommodate wearers ranging from infants through adults. In addition, it has been found that with the present invention, the densities and/or basis weights of the absorbent body 24 can be varied. In a particular aspect of the invention, the absorbent body 24 has an absorbent capacity of at least about 300 grams of synthetic urine.

In aspects wherein the absorbent body 24 includes the combination of hydrophilic fibers and high-absorbency particles, the hydrophilic fibers and high-absorbency particles can form an average basis weight for the absorbent body 24 that is within the range of about 400-900 grams per square meter. In certain aspects of the invention, the average composite basis weight of such an absorbent body 24 is within the range of about 500-800 grams per square meter, and preferably is within the range of about 550-750 grams per square meter to provide the desired performance.

To provide the desired thinness dimension to the various configurations of the absorbent article of the invention, the absorbent body 24 can be configured with a bulk thickness that is not more than about 0.6 centimeters. Preferably, the bulk thickness is not more than about 0.53 centimeters, and more preferably is not more than about 0.5 centimeters to provide improved benefits. The bulk thickness is determined under a restraining pressure of 0.2 psi (1.38 kPa).

The high-absorbency material can be selected from natural, synthetic, and modified natural polymers and materials. The high-absorbency materials can be inorganic materials, such as silica gels, or organic compounds, such as crosslinked polymers. The term “crosslinked” refers to methods for effectively rendering normally water-soluble materials substantially water insoluble but swellable. Such methods include, for example, physical entanglement, crystalline domains, covalent bonds, ionic complexes and associations, hydrophilic associations such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinyl morpholinone), poly(vinyl alcohol), and mixtures and copolymers thereof. Further polymers suitable for use in the absorbent body 24 include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and the like. Mixtures of natural and wholly or partially synthetic absorbent polymers can also be useful in the present invention.

The high absorbency material may be in any of a wide variety of geometric forms. As a general rule, it is preferred that the high absorbency material be in the form of discrete particles. However, the high absorbency material may also be in the form of fibers, flakes, rods, spheres, needles or other form that performs the intended absorbent function. In general, the high absorbency material is present in the absorbent body 24 in an amount of from about 5 to about 90 percent by weight, desirably in an amount of at least about 30 percent by weight, and even more desirably in an amount of at least about 50 percent by weight based on a total weight of the absorbent body 24. For example, in a particular aspect, the absorbent body 24 may include a laminate which includes at least about 50 percent by weight and desirably at least about 70 percent by weight of high-absorbency material overwrapped by a fibrous web or other suitable material for maintaining the high-absorbency material in a localized area.

An example of high-absorbency material suitable for use in the present invention is SANWET IM 3900 polymer available from Hoechst Celanese, a business having offices in Portsmouth, Va. Other suitable superabsorbents may include FAVOR SXM 880 polymer obtained from Stockhausen, a business having offices in Greensboro, N.C.

Optionally, a substantially hydrophilic tissue wrapsheet (not illustrated) may be employed to help maintain the integrity of the structure of the absorbent body 24. The tissue wrapsheet is typically placed about the absorbent body 24 over at least the two major facing surfaces thereof. The tissue wrapsheet can be composed of an absorbent cellulosic material, such as creped wadding or a high wet-strength tissue. In one aspect of the invention, the tissue wrapsheet can be configured to provide a wicking layer that helps to rapidly distribute liquid over the mass of absorbent fibers constituting the absorbent body 24.

The absorbent body 24 of the different aspects of the present invention further includes a plurality of zones of high air permeability which allow air and vapors to readily pass through the absorbent body 24 and through the vapor permeable outer cover 20 out of the diaper 10 into ambient air. For example, the absorbent body 24 may include a plurality of air passageways that provide the absorbent body 24 with zones or regions of high air permeability. The portions of the absorbent body 24 adjacent the air passageways provide zones or regions of high absorption. The zones of high air permeability are designed to provide the maximum air exchange from the absorbent body 24 while the zones of high absorption are designed to receive and hold the majority of the body exudates. The absorbent body 24 may define any number of zones of high air permeability that provide the improved air exchange. Desirably, the absorbent body 24 defines at least 3 and more desirably at least 5 different zones of high air permeability for improved performance.

The zones of high air permeability, such as the air passageways, are configured to enhance the breathability of the article to reduce the hydration of the wearer's skin during use without allowing excessive condensation of vapor, such as urine, on the garment facing surface of the outer cover 20. Such condensation of vapor on the outer surface of the diaper 10 can undesirably dampen the wearer's clothes. The zones of high air permeability are generally located in the area of the diaper over which air and vapor can transfer from the bodyside liner 22, through the absorbent body 24 and any other intervening layer or layers of material, and out the vapor permeable outer cover 20. For example, the zones of high air permeability may be located throughout the entire absorbent body 24 or may be selectively located in those regions of the absorbent body 24 that provide the maximum air exchange, such as the intermediate section 16 of the diaper 20. In a particular aspect, the zones of high air permeability are located in the front and intermediate sections 12 and 16, respectively, of the diaper 10 for improved air exchange.

The zones of high absorption, on the other hand, are not designed to transfer a high level of air and vapor from the interior of the diaper 10. Thus, the air exchange from the bodyside liner 22 of the diaper 10 to the outer cover 20 of the diaper and into the ambient atmosphere (exterior of the diaper 10) occurs generally through the absorbent body 24 in the zones of high air permeability. Some air exchange through the absorbent body 24 can also occur in the zones of high absorption to a limited degree. The zones of high air permeability may have any desired configuration including rectangular, circular, hourglass, oval, and the like, and may also include selected longitudinal or lateral strips or multiple regions which may be intermittently located.

The zones of high air permeability may have any desired dimensions that effectively provide improved air exchange while preventing excessive condensation of vapor from the absorbent body 24 through and onto the garment facing surface of the outer cover 20. Desirably, the zones of high air permeability may define a total area of from about 5 to about 75 percent, more desirably at least about 10 percent, even more desirably from about 10 to about 70 percent, and still more desirably from about 10 to about 60 percent of the total surface area of the absorbent body 24 of the diaper 10. For example, in a diaper 10 intended for use on a medium sized infant, the zones of high air permeability may define a total area of from about 6 to about 90 square centimeters.

When the total area of the zones of high air permeability is greater than the above amounts, the diaper 10 may exhibit an undesirable amount of condensation of vapor on the exposed, garment facing surface of the outer cover 20 undesirably resulting in a clammy feeling on the outer surface of the diaper 10. Whereas, when the total area of the zones of high air permeability is less than the above amounts, the diaper 10 may exhibit a low level of air exchange resulting in high levels of skin hydration that can undesirably lead to skin irritation and rash.

The zones of high air permeability of the absorbent body 24 of the diaper 10 are constructed to be substantially permeable to at least air and preferably permeable to water vapor. For example, the zones of high air permeability of the absorbent body 24 define a Frazier Porosity value which is at least about 10 percent, more desirably at least about 20 percent and even more desirably at least about 50 percent greater than the Frazier Porosity value of the zones of high absorption of the absorbent body 24. As used herein, the term “Frazier Porosity” refers to the value determined according to the Frazier Porosity Test method described in U.S. Pat. No. 6,217,890 issued Apr. 17, 2001 to Paul et al. When the zones of high air permeability exhibit Frazier Porosity values less than those indicated above, the diaper 10 may exhibit a low level of air exchange resulting in high levels of skin hydration that can undesirably lead to skin irritation and rash.

The zones of high air permeability may be provided in a variety of ways. The zones of high air permeability may be integral portions of the absorbent body 24 of the absorbent article or may be provided by apertures, holes or open spaces in the absorbent body 24. For example, portions of the absorbent body 24 may be discontinuous or removed to provide the zones. Alternatively, the zones of high air permeability may be provided by portions of the absorbent body 24 that are constructed to absorb less fluid exudates thereby resulting in improved air flow through such portions in use. For example, portions of the absorbent body 24 may be void of or contain substantially less high-absorbency material than other portions of the absorbent body 24 to provide such improved air flow. Portions of the absorbent body 24 may otherwise be treated or coated with a solution that renders them hydrophobic to provide the zones of high air permeability in selected areas. In other alternative configurations, the zones of high air permeability may be provided by creating voids or holes in the absorbent body 24 and placing other materials having a higher air permeability than the absorbent body 24, such as those materials described below as being suitable for the surge management layer 34, in the holes or voids.

Due to the thinness of absorbent body 24 and the high absorbency material within the absorbent body 24, the liquid uptake rates of the absorbent body 24, by itself, may be too low, or may not be adequately sustained over multiple insults of liquid into the absorbent body 24. To improve the overall liquid uptake and air exchange, the diaper 10 of the different aspects of the present invention may further include a porous, liquid-permeable layer of surge management material 34, as representatively illustrated in FIG. 1. The surge management layer 34 is typically less hydrophilic than the absorbent body 24, and has an operable level of density and basis weight to quickly collect and temporarily hold liquid surges, to transport the liquid from its initial entrance point and to substantially completely release the liquid to other parts of the absorbent body 24. This configuration can help prevent the liquid from pooling and collecting on the portion of the diaper 10 positioned against the wearer's skin, thereby reducing the feeling of wetness by the wearer. The structure of the surge management layer 34 also generally enhances the air exchange within the diaper 10.

Various woven and nonwoven fabrics can be used to construct the surge management layer 34. For example, the surge management layer 34 may be a layer composed of a meltblown or spunbond web of synthetic fibers, such as polyolefin fibers. The surge management layer 34 may also be a bonded-carded-web or an airlaid web composed of natural and synthetic fibers. The bonded-carded-web may, for example, be a thermally bonded web that is bonded using low melt binder fibers, powder or adhesive. The webs can optionally include a mixture of different fibers. The surge management layer 34 may be composed of a substantially hydrophobic material, and the hydrophobic material may optionally be treated with a surfactant or otherwise processed to impart a desired level of wettability and hydrophilicity. In a particular aspect, the surge management layer 34 includes a hydrophobic, nonwoven material having a basis weight of from about 30 to about 120 grams per square meter.

For example, in a particular aspect, the surge management layer 34 may include a bonded-carded-web, nonwoven fabric that includes bicomponent fibers and that defines an overall basis weight of about 83 grams per square meter. The surge management layer 34 in such a configuration can be a homogeneous blend composed of about 60 weight percent polyethylene/polyester (PE/PET), sheath-core bicomponent fibers that have a fiber denier of about 3 d and about 40 weight percent single component polyester fibers that have a fiber denier of about 6 d and that have fiber lengths of from about 3.8 to about 5.08 centimeters.

In the illustrated aspects, the surge management layer 34 is arranged in a direct, contacting liquid communication with the absorbent body 24. The surge management layer 34 may be operably connected to the bodyside liner 22 with a conventional pattern of adhesive, such as a swirl adhesive pattern. In addition, the surge management layer 34 may be operably connected to the absorbent body 24 with a conventional pattern of adhesive. The amount of adhesive add-on should be sufficient to provide the desired levels of bonding, but should be low enough to avoid excessively restricting the movement of liquid from the bodyside liner 22, through the surge management layer 34 and into the absorbent body 24.

The absorbent body 24 is positioned in liquid communication with surge management layer 34 to receive liquids released from the surge management layer 34, and to hold and store the liquid. In the shown aspect, the surge management layer 34 includes a separate layer that is positioned over another, separate layer including the absorbent body 24, thereby forming a dual-layer arrangement. The surge management layer 34 serves to quickly collect and temporarily hold discharged liquids, to transport such liquids from the point of initial contact and spread the liquid to other parts of the surge management layer 34, and then to substantially completely release such liquids into the layer or layers constituting the absorbent body 24.

The surge management layer 34 can be of any desired shape. Suitable shapes include for example, circular, rectangular, triangular, trapezoidal, oblong, dog-boned, hourglass-shaped, or oval. In certain aspects, for example, the surge management layer 34 can be generally rectangular-shaped. In the illustrated aspects, the surge management layer 34 is coextensive with the absorbent body 24. Alternatively, the surge management layer 34 may extend over only a part of the absorbent body 24. Where the surge management layer 34 extends only partially along the length of the absorbent body 24, the surge management layer 34 may be selectively positioned anywhere along the absorbent body 24. For example, the surge management layer 34 may function more efficiently when it is offset toward the front waist section 12 of the diaper 10. The surge management layer 34 may also be approximately centered about the longitudinal center line of the absorbent body 24.

Additional materials suitable for the surge management layer 34 are set forth in U.S. Pat. No. 5,486,166 issued Jan. 23, 1996 in the name of C. Ellis et al. and entitled “FIBROUS NONWOVEN WEB SURGE LAYER FOR PERSONAL CARE ABSORBENT ARTICLES AND THE LIKE”; U.S. Pat. No. 5,490,846 issued Feb. 13, 1996 in the name of Ellis et al. and entitled “IMPROVED SURGE MANAGEMENT FIBROUS NONWOVEN WEB FOR PERSONAL CARE ABSORBENT ARTICLES AND THE LIKE”; and U.S. Pat. No. 5,364,382 issued Nov. 15, 1994 in the name of Latimer et al. and entitled “ABSORBENT STRUCTURE HAVING IMPROVED FLUID SURGE MANAGEMENT AND PRODUCT INCORPORATING SAME”, the disclosures of which are hereby incorporated by reference.

As representatively illustrated in FIG. 1, the diaper 10 may also include a ventilation layer 32 located between the outer cover 20 and the absorbent body 24. The ventilation layer 32 serves to facilitate the movement of air within and through the diaper 10 and prevent the outer cover 20 from being in surface to surface contact with at least a portion of the absorbent body 24. Specifically, the ventilation layer 32 serves as a conduit through which air and water vapor can move from the absorbent body 24 through the vapor permeable outer cover 20.

The ventilation layer 32 may be formed from materials described above as being suitable for the surge management layer 34 such as nonwoven, (e.g., spunbond, meltblown or carded), woven, or knitted fibrous webs composed of natural fibers and/or synthetic polymeric fibers. Suitable fibers include, for example, acrylic fibers, polyolefin fibers, polyester fibers, or blends thereof. The ventilation layer 32 may also be formed from a porous foam material such as an open-celled polyolefin foam, a reticulated polyurethane foam, and the like. The ventilation layer 32 may include a single layer of material or a composite of two or more layers of material. In a particular aspect, the ventilation layer 32 includes a hydrophobic, nonwoven material having a thickness of at least about 0.10 centimeters determined under a restraining pressure of 0.05 psi (0.34 kPa) and a basis weight of from about 20 to about 120 grams per square meter. For example, the ventilation layer 32 may include a bonded-carded-web, nonwoven fabric that includes bicomponent fibers and that defines an overall basis weight of about 83 grams per square meter. The ventilation layer 32 in such a configuration can be a homogeneous blend composed of about 60 weight percent polyethylene/polyester (PE/PET), sheath-core bicomponent fibers that have a fiber denier of about 3 d and about 40 weight percent single component polyester fibers that have a fiber denier of about 6 d and that have fiber lengths of from about 3.8 to about 5.08 centimeters.

The ventilation layer 32 can be of any desired shape. Suitable shapes include for example, circular, rectangular, triangular, trapezoidal, oblong, dog-boned, hourglass-shaped, or oval. The ventilation layer 32 may extend beyond, completely over or partially over the absorbent body 24. For example, the ventilation layer 32 may suitably be located over the intermediate section 16 of the diaper 10 and be substantially centered side-to-side with respect to the longitudinal centerline 36 of the diaper 10. It is generally desired that the entire absorbent body 24 be overlaid with the ventilation layer 32 to prevent substantially all surface to surface contact between the outer cover 20 and the absorbent body 24. In the illustrated aspects, the ventilation layer 32 is coextensive with the absorbent body 24. This allows for the maximum degree of air exchange with minimal dampness on the garment facing surface of the outer cover 20.

In the illustrated aspects, the ventilation layer 32 is arranged in a direct, contacting liquid communication with the absorbent body 24. The ventilation layer 32 may be operably connected to the outer cover 20 with a conventional pattern of adhesive, such as a swirl adhesive pattern. In addition, the ventilation layer 32 may be operably connected to the absorbent body 24 with a conventional pattern of adhesive. The amount of adhesive add-on should be sufficient to provide the desired levels of bonding, but should be low enough to avoid excessively restricting the movement of air and vapor from the absorbent body 24 and through the outer cover 20.

The ventilation layer 32 may further serve to quickly collect and temporarily hold discharged liquids, which pass through the absorbent body 24 and, in particular, through the zones of high air permeability within the absorbent body 24. The ventilation layer 32 may then transport such liquids from the point of initial contact and spread the liquid to other parts of the ventilation layer 32, and then substantially completely release such liquids into the layer or layers of the absorbent body 24.

In order to protect the barrier of the skin covered by the diaper 10, a composition is applied to the bodyfacing surface 11 of the bodyside liner 22 of the diaper 10. The composition generally can include emollient(s), viscosity enhancer(s) and silicone elastomer(s). The composition can also include natural fats or oils, solidifying agents and sterols or sterol derivatives. For example, the compositions of the invention may include from about 40 to about 95 percent by weight of one or more emollients; from about 0.1 to about 40 percent by weight of one or more viscosity enhancers; and, from about 0.1 to about 20 percent by weight of one or more silicone elastomers. The composition may include other ingredients as well. Ranges are used to describe the relative amounts of components in the compositions of the invention as well as to describe the relative physical properties of the compositions. These ranges are illustrative and one of skill in the art will recognize that the nature of the composition will dictate the various levels of components that must be used to achieve the intended benefit for the skin barrier. The levels can be determined by routine experimentation in view of the disclosure provided herein.

The compositions of the invention can be in a variety of physical forms including emulsions, lotions, creams, ointments, salves, suspensions, gels or hybrids of these forms.

The emollients of the compositions act as lubricants to reduce the abrasiveness of the bodyside liner 22 to the skin and, upon transfer to the skin, help to maintain the soft, smooth and pliable appearance of the skin. Emollients are skin conditioning ingredients that help to soften, smooth, plasticize, lubricate, moisturize, improve the appearance of, improve the feel of and protect the skin. Suitable emollients that can be incorporated into the compositions include, but are not limited to, oils such as petroleum based oils, petrolatum, vegetable based oils, hydrogenated vegetable oils, animal oils, hydrogenated animal oils, mineral oils, natural or synthetic oils, alkyl dimethicones, alkyl methicones, alkyldimethicone copolyols, phenyl silicones, alkyl trimethylsilanes, dimethicone, lanolin and its derivatives, esters, branched esters, glycerol esters and derivatives, propylene glycol esters and derivatives, alkoxylated carboxylic acids, alkoxylated alcohols, fatty alcohols, triglycerides, alkyl hydroxystearates and mixtures of such compounds. The esters can be selected from, but are not limited to, cetyl palmitate, stearyl palmitate, cetyl stearate, isopropyl laurate, isopropyl myristate, isopropyl palmitate and mixtures thereof. Ethers such as eucalyptol, cetearyl glucoside, dimethyl isosorbicide polyglyceryl-3 cetyl ether, polyglyceryl-3 decyltetradecanol, propylene glycol myristyl ether and mixtures thereof can also be used as emollients. The fatty alcohols include octyldodecanol, lauryl, myristyl, cetyl, stearyl and behenyl alcohol and mixtures thereof. For example, a particularly well suited emollient is petrolatum. Other conventional emollients may also be added in a manner that maintains the desired properties of the compositions set forth herein.

To provide stability and transfer to the skin of the wearer, the compositions may include from about 40 to about 95 percent by weight and desirably from about 70 to about 80 percent by weight of one or more emollients. In particular aspects, the emollient can be at least a minimum of about 40 percent by weight. The emollient can alternatively be at least about 70 percent, and optionally, can be at least about 80 percent to provide improved performance. In other aspects, the emollient can be not more than a maximum of about 95 percent by weight. Compositions that include an amount of emollient less than the recited amounts tend to provide less transfer to the wearer's skin.

One or more viscosity enhancers may be added to the composition to increase the viscosity, to help stabilize the formulation on the bodyfacing surface 11 of the bodyside liner 22 and, thereby, to reduce migration and improve transfer to the skin. The viscosity enhancer increases the meltpoint viscosity of the compositions to have a high viscosity (greater than about 50,000 centipoise) under low shear at the “hot box car” stability temperature of about 54.5° C. and at lower temperatures. Having viscosity at elevated temperatures prevents the compositions from migrating into or away from the materials to which they are applied. However, the viscosity enhancer component also provides a low viscosity (less than about 5,000 centipoise) under shear for the compositions at process conditions. Typically, process temperatures are approximately 5° C. above the melting point of the composition. Generally, the process temperature is about 60° C. or higher. Different compositions of the invention will have different melting points. The viscosity enhancers of the invention are capable of maintaining the viscosity of compositions of the invention up to temperatures just below the desired processing temperature for a given composition. Examples of suitable viscosity enhancers include polyolefin resins, lipophilic/oil thickeners, ethylene/vinyl acetate copolymers, natural clays, synthetic analogs of natural clays, organically modified clays, quaternary modified clays, quaternary starch compounds, polyethylene, silica, silica silylate, silica methyl silylate, colloidal silicone dioxide, cetyl hydroxy ethyl cellulose, other organically modified celluloses, PVP/decane copolymer, PVM/MA decadiene crosspolymer, PVP/eicosene copolymer, PVP/hexadecane copolymer, microcrystalline wax, hexadecyl-cosanyl-hexacosanate, shellac wax, glycol montanate, PEG-12 carnauba, synthetic paraffin, ozokerite, C₂₀-C₄₀ alkyl hydroxystearyl stearate, polyperfluoromethylisopropylether montan wax and mixtures of these compounds. A particularly well suited viscosity enhancer is an ethylene/vinyl acetate copolymer commercially available from E. I. DuPont (Wilmington, Del.) under the trade designation “ELVAX”.

To provide the improved transfer to the skin of the wearer, the composition may include from about 0.1 to about 40 percent by weight, desirably from about 5 to about 20 percent by weight, and more desirably from about 10 to about 15 percent by weight of the viscosity enhancer for reduced migration and improved transfer to the wearer's skin. In particular aspects, the viscosity enhancer can be at least a minimum of about 0.1 percent by weight. The viscosity enhancer can alternatively be at least about 5 percent, and optionally, can be at least about 10 percent to provide improved performance. In other aspects, the viscosity enhancer can be not more than a maximum of about 40 percent by weight. The viscosity enhancer can alternatively be not more than about 20 percent, and optionally, can be not more than about 15 percent to provide improved effectiveness.

Additionally, the compositions of the invention may include from about 0.1 to about 20 percent by weight of silicone elastomer. The silicone elastomers contribute to the improved rheology of the compositions, contribute to the compositions being breathable and give the compositions an anti-irritant effect. More particularly, the compositions of the invention include from about 5 to about 15 percent by weight of one or more silicone elastomers. In particular aspects, the silicone elastomer can be at least a minimum of about 0.1 percent by weight and can alternatively be at least about 5 percent by weight. In other aspects, the silicone elastomer can be not more than a maximum of about 20 percent by weight and can alternatively be not more than about 15 percent.

Examples of suitable silicone elastomers include, but are not limited to, crosslinked non-emulsifying siloxane elastomers formed from a divinyl compound reacted with Si—H linkages of a polysiloxane; crosslinked non-emulsifying siloxane elastomers formed from a C₃-C₂₀ alkyl polysiloxane compound reacted with Si—H linkages of a polysiloxane and mixtures of these compounds. Examples of suitable crosslinked non-emulsifying siloxane elastomers include Vinyl Dimethicone/Methicone Crosspolymer, Crosslinked Stearyl Methyl Dimethyl Siloxane Copolymer, Dimethicone/Vinyl Dimethicone Crosspolymer, Dimethicone/Phenyl Vinyl Dimethicone Crosspolymer and mixtures of these compounds. Examples of suitable emulsifying silicone elastomers include vinyl MQ resin/organopolysiloxane crosspolymers where the organo group is a polyglycol, polyglycerol, oligosaccharide, hydroxyl-terminated polyoxyalkylene polyether and their carboxylate esters, and lower alkanol ethers, and mixtures of these compounds. Additional suitable silicone elastomers are described in U.S. Pat. No. 5,849,314 issued Dec. 15, 1998 to Dobkowski et al., incorporated herein by reference. Further, suitable silicone elastomers are also described in European Patent Application 1 057 476 Al published Dec. 6, 2000, entitled “Organopolysiloxane Gels for Use in Cosmetics” and assigned to Wacker-Chemie GmbH.

The compositions of the invention can also include one or more solidifying agents. The solidifying agent(s) in the compositions of the present invention can function to help solidify the composition so that the composition is a solid at room temperature and has a penetration hardness of at least 5 mm and has a melting point of at least 32° C. The solidifying agent may also provide a tackiness to the composition that improves the transfer by adhesion to the skin of the wearer. Depending on the solidifying agent selected, the solidifying agent can also modify the mode of transfer so that the composition tends to fracture or flake off instead of actually rubbing off onto the skin of the wearer which can lead to improved transfer to the skin. The solidifying agent may further function as an emollient, occlusive agent, moisturizer, barrier enhancer, viscosity enhancer and combinations thereof. The solidifying agents may include waxes as well as compounds that perform functionally as waxes.

The solidifying agents can be selected from alkyl siloxanes, polymers, hydrogenated vegetable oils having a melting point of 35° C. or greater, fatty acid esters with a melting point of 35° C. or greater, alkyl hydroxystearates (>C₁₆), branched esters, alkoxylated alcohols and alkoxylated carboxylic acid. Additionally, the solidifying agents can be selected from animal, vegetable and mineral waxes and alkyl silicones. Examples of solidifying agents include, but are not limited to, the following: alkyl silicones, alkyl trimethylsilanes, beeswax, behenyl behenate, behenyl benzoate, C₂₄-C₂₈ alkyl dimethicone, C₃₀ alkyl dimethicone, cetyl methicone, stearyl methicone, cetyl dimethicone, stearyl dimethicone, cerotyl dimethicone, candelilla wax, carnuba, synthetic carnuba, PEG-12 carnauba, cerasin, hydrogenated microcrystalline wax, jojoba wax, microcrystalline wax, lanolin wax, ozokerite, paraffin, synthetic paraffin, cetyl esters, behenyl behenate, C₂₀-C₄₀ alkyl behenate, C₁₂-C₁₅ lactate, cetyl palmitate, stearyl palmitate, isosteryl behenate, lauryl behenate, stearyl benzoate, behenyl isostearate, cetyl myristate, cetyl octanoate, cetyl oleate, cetyl ricinoleate, cetyl stearate, decyl oleate, di-C₁₂-C₁₅ alkyl fumerate, dibehenyl fumerate, myristyl lactate, myristyl lignocerate, myristyl myristate, myristyl stearate, lauryl stearate, octyldodecyl stearate, octyldodecyl stearoyl stearate, oleyl arachidate, oleyl stearate, tridecyl behenate, tridecyl stearate, tridecyl stearoyl stearate, pentaerythrityl tetrabehenate, pentaerythritylhydrogenated rosinate, pentaerythrityl distearate, pentaerythrityl tetraabeite, pentaerythrityl tetracocoate, pentaerythrityl tetraperlargonate, pentaerythrityl tetrastearate, ethylene vinyl acetate, polyethylene, hydrogenated cottonseed oil, hydrogenated vegetable oil, hydrogenated squalene, hydrogenated coconut oil, hydrogenated jojoba oil, hydrogenated palm oil, hydrogenated palm kernel oil, hydrogenated olive oil, polyamides, metal stearates and other metal soaps, C₃₀-C₆₀ fatty alcohols, C₂₀₊ fatty amides, polypropylene, polystyrene, polybutane, polybutylene terephthalate, polydipentane, polypropylene, zinc stearate, dodecyl laurate, stearyl palmitate, octadecyl hexadecanoate, octadecyl palmitate, stearyl behenate, docosyl octanoate, tetradecyl-octadecanyl behenate, hexadecyl-cosanyl hexacosanate, shellac wax, glycol montanate, fluoranated waxes, C₂₀-C₄₀ alkyl hydroxystearyl stearate and mixtures of such compounds and mixtures of such compounds. In one aspect, the solidifying agent is a blend including about 70 weight percent cerasin wax, about 10 weight percent microcrystalline wax, about 10 weight percent paraffin wax and about 10 weight percent cetyl esters (synthetic spermaceti wax).

To provide improved transfer to the skin of the wearer, the composition may include from about 5 to about 60 percent by weight and desirably from about 25 to about 50 percent by weight of solidifying agent(s). In particular aspects, the solidifying agent can be at least a minimum of about 5 percent by weight. The solidifying agent can alternatively be at least about 25 percent to provide improved performance. In other aspects, the solidifying agent can be not more than a maximum of about 60 percent by weight. The solidifying agent can alternatively be not more than about 50 percent to provide improved effectiveness. Compositions that include an amount of solidifying agent less than the recited amounts tend to be too soft and may have lower viscosities that may undesirably lead to migration of the composition away from bodyfacing surfaces 11 of the absorbent article, thus diminishing transfer to the wearer's skin. Whereas, compositions that include an amount of solidifying agent greater than the recited amounts tend to provide less transfer to the wearer's skin.

The compositions of the invention can further include fats and oils. The fats and oils can provide a source of essential and non-essential fatty acids similar to those found in the skin's natural barrier. Fats and oils include compounds that are fats, oils, essential oils, fatty acids, fatty alcohols, phospholipids and mixtures of such compounds. Fats and oils include oils derived from plant and animal sources. Similarly, the essential oils include essential oils derived from plant sources. Those of skill in the art would understand that all compounds commonly understood to have the structure of or to function as fats, oils, essential oils, fatty acids, fatty alcohols and phospholipids can be used as the natural fat or oil component of the composition of the invention. While an exhaustive list of each and every fat and oil that could be used in the compositions of the invention is not provided, those of skill in the art will understand and appreciate the individual compounds that can serve as a fat or oil component of the compositions of the invention.

Representative examples of fats and oils include, but are not limited to: Avocado Oil, Apricot Oil, Babassu Oil, Borage Oil, Camellia Oil, Canola Oil, Castor Oil, Coconut Oil, Corn Oil, Cottonseed Oil, Evening Primrose Oil, Hydrogenated Cottonseed Oil, Hydrogenated Palm Kernel Oil, Maleated Soybean Oil, Meadowfoam Oil, Palm Kernel Oil, Peanut Oil, Rapeseed Oil, Safflower Oil, Sphingolipids, Sweet Almond Oil, Tall Oil, Lanolin, Lanolin Alcohol, Lauric Acid, Palmitic Acid, Stearic Acid, Linoleic Acid, Stearyl Alcohol, Lauryl Alcohol, Myristyl Alcohol, Behenyl Alcohol, Rose Hip Oil, Calendula Oil, Chamomile Oil, Eucalyptus Oil, Juniper Oil, Sandlewood Oil, Tea Tree Oil, Sunflower Oil, and Soybean Oil. Another suitable fat/oil for the compositions of the invention is PROLIPID 141 blend available from International Specialty Products of Wayne, N.J. The PROLIPID 141 blend is a mixture of glyceryl stearate, fatty acids, fatty alcohols and phospholipids.

In order to assist in replenishing skin barrier enhancing agents, the compositions of the invention may include fats and oils in an amount of from about 0.1 to about 59 percent by weight and desirably from about 10 to about 50 percent by weight. In particular aspects, the fats and oils can be at least a minimum of about 0.1 percent by weight. The fats and oils can alternatively be at least about 10 percent to provide improved performance. In other aspects, the fats and oils can be not more than a maximum of about 59 percent by weight. The fats and oils can alternatively be not more than about 50 percent to provide improved effectiveness.

The compositions of the invention also include sterols and sterol derivatives that act in combination with the natural fats/oils to provide natural skin barrier enhancement and skin barrier recovery. Sterols and sterol derivatives that can be used in the compositions of the invention include, but are not limited to: β-sterols having a tail on the 17 position and having no polar groups for example, cholesterol, sitosterol, stigmasterol, and ergosterol, as well as, C₁₀-C₃₀ cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyldecanoate, dihydrolanosterol, dihydrolanosteryl octyldecanoate, ergocalciferol, tall oil sterol, soy sterol acetate, lanasterol, soy sterol, avocado sterols, “AVOCADIN” (trade name of Croda Ltd of Parsippany, N.J.), sterol esters and similar compounds, as well as mixtures thereof. The compositions of the invention can include sterols, sterol derivatives or mixtures of both sterols and sterol derivatives in an amount of from about 0.1 to about 10 percent by weight, desirably from about 0.5 to about 5 percent by weight and more desirably from about 0.8 to about 1 percent by weight of the composition. In particular aspects, the sterols can be at least a minimum of about 0.1 percent by weight. The sterols can alternatively be at least about 0.5 percent, and optionally, can be at least about 0.8 percent to provide improved performance. In other aspects, the sterols can be not more than a maximum of about 10 percent by weight. The sterols can alternatively be not more than about 5 percent, and optionally, can be not more than about 1 percent to provide improved effectiveness.

If it is desired that the composition provide a treatment for the skin, the composition can also include an active ingredient such as a diaper rash skin protectant. Skin protectants are drug products that protect injured or exposed skin or mucous membrane surface from harmful or annoying stimuli. Suitable active ingredients, in addition to those mentioned above as suitable emollients, that can be incorporated into the composition include, but are not limited to, allantoin and its derivatives, aloe, aluminum hydroxide gel, calamine, cocoa butter, dimethicone, cod liver oil, glycerin, kaolin and its derivatives, lanolin and its derivatives, mineral oil, petrolatum, shark liver oil, talc, topical starch, zinc acetate, zinc carbonate, zinc oxide and the like, and mixtures thereof. The composition may include from about 0.10 to about 59 percent by weight of the active ingredient depending upon the skin protectant, the amount desired to be transferred to the skin or the amount of a particular skin protectant required in the U.S. Food and Drug Administration monograph.

In order to better enhance the benefits to the wearer, additional ingredients can be included in the compositions of the present invention. For example, the classes of ingredients that may be used and their corresponding benefits include, without limitation: antifoaming agents (reduce the tendency of foaming during processing); antimicrobial actives; antifungal actives; antiseptic actives; antioxidants (product integrity); antioxidants-cosmetic (reduce oxidation); astringents-cosmetic (induce a tightening or tingling sensation on skin); astringent-drug (a drug product that checks oozing, discharge, or bleeding when applied to skin or mucous membrane and works by coagulating protein); biological additives (enhance the performance or consumer appeal of the product); colorants (impart color to the product); deodorants (reduce or eliminate unpleasant odor and protect against the formation of malodor on body surfaces); other emollients (help to maintain the soft, smooth, and pliable appearance of the skin by their ability to remain on the skin surface or in the stratum corneum to act as lubricants, to reduce flaking, and to improve the skin's appearance); external analgesics (a topically applied drug that has a topical analgesic, anesthetic, or antipruritic effect by depressing cutaneous sensory receptors, or that has a topical counterirritant effect by stimulating cutaneous sensory receptors); film formers (to hold active ingredients on the skin by producing a continuous film on skin upon drying); fragrances (consumer appeal); silicones/organomodified silicones (protection, water resistance, lubricity, softness); oils (mineral, vegetable, and animal); natural moisturizing agents (NMF) and other skin moisturizing ingredients known in the art; opacifiers (reduce the clarity or transparent appearance of the product); powders (enhance lubricity, oil adsorption, provide skin protection, astringency, opacity, etc.); skin conditioning agents; solvents (liquids employed to dissolve components found useful in the cosmetics or drugs); and surfactants (as cleansing agents, emulsifying agents, solubilizing agents, and suspending agents).

An important property of the compositions of the different aspects of the present invention is their ability to remain on the surface of the bodyside liner 22 and their resistance to migration into the diaper 10 such that they can readily be transferred to the wearer's skin. In this regard, the articles having the compositions of the present invention applied to their bodyside liner 22 define a z-direction migration loss of no more than about 55%, desirably no more than about 50%, more desirably no more than about 45%, even more desirably no more than about 40% and yet even more desirably no more than about 35% when subjected to the Z-Direction Lotion Migration Test described in U.S. Pat. No. 6,149,934 issued Nov. 21, 2000 to Krzysik et al. incorporated herein by reference. In articles that have a greater z-direction migration loss, the composition undesirably migrates into the interior and along the surface of the bodyside liner 22 and at times through the bodyside liner 22 into the absorbent body 24 of the article which results in a lower reduction in abrasion and less transfer to the skin of the wearer.

Moreover, to provide the improved stability and transfer to the skin of the wearer, the compositions of the present invention may define a melting point of from about 32° C. to about 100° C., desirably from about 35° C. to about 80° C., and more desirably from about 40° C. to about 75° C. Compositions that have lower melting points exhibit migration of the composition during use and at elevated temperatures in storage that can undesirably result in reduced transfer to the skin. Whereas, compositions that have higher melting points may require that the composition be at a temperature above the flash point of the bodyside liner 22 material which can undesirably lead to fires. The melting points of the compositions of the invention cause the compositions to be relatively immobile and localized on the bodyfacing surface 11 of the diaper 10 at room temperature and readily transferable to the skin of the wearer at body temperatures. However, the compositions of the invention are not completely liquid under extreme storage conditions. Desirably, the compositions are easily transferable to the skin by way of normal contact, wearer motion, adhesion or body heat. When the compositions are relatively immobilized at room temperature, a lesser quantity of composition is required on the bodyfacing surface 11 to provide a beneficial effect.

The composition of the present invention may further define a low shear viscosity at about 55° C. of greater than about 50,000 centipoise, desirably from about 50,000 to about 1,000,000 centipoise, and more desirably from about 100,000 to about 800,000 centipoise for reduced migration and improved transfer to the skin of the wearer. Compositions that have lower melt point viscosities exhibit migration of the composition through the bodyside liner 22 into the absorbent body 24 of the article which can undesirably result in reduced transfer to the skin. Whereas, compositions that have higher melt point viscosities may be so solid as to also exhibit a reduced transfer to the skin.

Further, to provide the improved stability and transfer to the skin of the wearer, the compositions of the present invention may also define a high shear viscosity of less than about 5,000 centipoise, desirably from about 100 to about 500 centipoise, and more desirably from about 150 to about 250 centipoise at a temperature of about 60° C. (or higher temperatures depending on the components and melting point of the composition).

The penetration hardness of the compositions of this invention can be from about 5 to about 365 millimeters, more desirably from about 10 to about 300 millimeters, more desirably from about 20 to about 200 millimeters, and still more desirably from about 40 to about 120 millimeters at 25° C. (Compositions having a needle penetration hardness greater than 365 millimeters cannot be measured using ASTM method D 1321). The hardness of the compositions of this invention is important for two reasons. First, the softer the formulation the more mobile the formulation will be, making the formulation more likely to migrate to the inner plies of the diaper 10, which is not desirable. Secondly, softer compositions tend to be more greasy/oily to the touch, which is also less desirable.

The composition may be applied to the entire bodyfacing surface 11 of the bodyside liner 22 or may be selectively applied to particular sections of the bodyfacing surface 11, such as the medial section along the longitudinal centerline of the diaper 10, to provide greater lubricity of such sections and to transfer such composition to the wearer's skin. Alternatively, the bodyfacing surface 11 of the bodyside liner 22 may include multiple stripes of the composition applied thereto. For example, the bodyfacing surface 11 of the bodyside liner 22 may include from 1 to 20 stripes of composition extending along the longitudinal direction of the diaper 10. The stripes may extend the full length of the bodyside liner 22 or only a portion thereof. The stripes may also define a width of from about 0.2 to about 1 centimeters.

The composition should cover a sufficient amount of the bodyfacing surface 11 of the bodyside liner 22 to ensure adequate transfer to the skin and reduced abrasion between the bodyside liner 22 and the wearer's skin. Desirably, the composition is applied to at least about 5 percent and more desirably at least about 25 percent of the bodyfacing surface 11 of the bodyside liner 22.

The composition can be applied to the bodyside liner 22 at any add-on level that provides the desired transfer benefit. For example, the total add-on level of the composition can be from about 0.05 to about 100 mg/cm², desirably from about 1 to about 50 mg/cm² and more desirably from about 10 to about 40 mg/cm² for improved performance. The add-on amount will depend upon the desired effect of the composition on the skin barrier function and the specific composition. As discussed above, the improved stability and reduced tendency to migrate of the compositions of the present invention allows a lesser amount of composition to be applied to the bodyside liner 22 to achieve the same benefit when compared with conventional compositions.

The composition may be applied to the bodyside liner 22 in any of many well known manners. A preferred method to uniformly apply the composition to the bodyfacing surface 11 of the bodyside liner 22 is spraying or slot coating. Spraying or slot coating the composition is the most exact process and offers maximum control of the composition distribution and transfer rate. However, other methods, such as rotogravure or flexographic printing and foam application can be used. The compositions of the present invention can be applied after the bodyfacing material has been incorporated into the absorbent article or prior to incorporating the body facing material into the absorbent article.

The composition may be applied to the bodyside liner 22 by (a) heating the composition to a temperature above the melting point of the composition, causing the composition to melt, (b) uniformly applying the melted composition to the bodyfacing surface 11 of the bodyside liner 22; and (c) resolidifying the composition applied to the bodyfacing surface 11. Desirably, resolidification of the composition occurs almost instantaneously, without the need for external cooling devices such as chill rolls. This can occur if the composition is heated to a temperature only slightly above or at the melting point of the composition. However, external cooling devices such as chill rolls, either before or after the application of melt, can be used if desired to accelerate resolidification. Other cooling methods such as cooling tunnels could also be used.

The increased viscosity of the composition at the process temperature and the instantaneous resolidification tends to impede penetration of the composition into the bodyside liner 22 and absorbent body 24 of the diaper 10 and retain it on the bodyfacing surface 11 of the bodyside liner 22, which is advantageous. For example, the temperature of the melted composition can advantageously be less than about 10° C., more desirably less than about 5° C., and still more desirably less than about 2° C. above the melting point of the composition prior to applying it to the bodyside liner 22 for reduced migration. As the temperature of the melted composition approaches the freezing point of the composition, the viscosity of the melted composition generally increases, which further enhances the tendency of the melted composition to be retained on the bodyfacing surface 11.

The compositions of the invention are believed to provide better protection to the skin barrier. In particular, the compositions of the invention have been shown to be capable of reducing the skin's inflammatory response to irritants. While not intending to be limited by theory, it is believed that the compositions reduce the irritation response by providing a better barrier against the irritants. Further, the compositions of the invention are believed to exhibit improved breathability. Breathability is a desirable trait of compositions applied to the bodyfacing surfaces of absorbent articles. Ideally, such compositions provide a good barrier to enhance the skin's natural barrier properties and remain breathable so as to not interfere with the skin's respiration.

In order to evaluate the ability to reduce irritation response by providing a better barrier, a human skin culture was selected to model the response of the human epidermis. EPIDERM skin culture is a cornified, air-interfaced human skin culture. EPIDERM skin culture has multiple layers of progressively differentiated keratinocytes resembling human epidermis. EPIDERM EPI-200 skin culture can be purchased from MatTek Corporation of Ashland, Mass. Experiments using EPIDERM skin culture are conducted in six well plates. Typically, five EPIDERM skin culture inserts are added to five of the six wells. Each well contains one milliliter of pre-warmed media that is the same as the EPIDERM skin culture media. The plates are then incubated in a 37° C., 5% CO₂ incubator for thirty minutes. After incubation, 15 microliters of test composition or control are applied to the surface of the EPIDERM skin culture after removing any residual media. For test compositions using a petrolatum base, the composition is applied using a positive-displacement pipettor and spread over the skin culture surface using a glass rod. The well plates, with the test compositions/control applied, are incubated in the 37° C., 5% CO₂ incubator for thirty minutes after which the underlying media is removed and replaced with fresh, pre-warmed media. Next, ten microliters of insult solution, either fecal protease or bile acid, are applied to the surface of the EPIDERM skin culture.

Infant feces contain proteases that include trypsin and chymotrypsin (See Haverback, B. J., Dyce, B. J., Gutentag, P. J., and Montgomery, D. W. (1963) Measurement of Trypsin and Chymotrypsin in Stool. Gastroenterology 44:588-597; and Barbero, G. J., Sibinga, M. S., Marino, J. M., and Seibel, R. (1966) Stool Trypsin and Chymotrypsin. Amer. J. Dis. Child 112:536-540). For internal studies, infant feces were collected and the amount of total protease and trypsin activities determined for each of the fecal extracts. To prepare the extract, the feces were suspended in water and vigorously vortexed. After vortexing, the samples were held on ice prior to centrifugation at 15,000 times the force of gravity for 20 minutes. The supernatant was filtered through 0.22 micron cellulose acetate filters and stored at −80° C. until use. The amount of trypsin activity in the fecal extracts ranged from 0.4-402 μg/ml (n=19) as measured by the ability of the sample to hydrolyze a fluorescently-labeled trypsin peptide substrate (Boc-Gln-Ala-Arg-AMC HCl, BACHEM California, Incorporated, Torrance, Calif.). Total protease activity was measured as the ability of the sample to hydrolyze a fluorescent dye-labeled casein substrate (EnzChek Protease Assay Kit (E-6639), Molecular Probes, Eugene, Oreg.). Irritation induced in the EPIDERM skin culture correlated with the total protease as well as trypsin activities of the fecal extracts. Based on the literature sources as well as internal data, a trypsin-chymotrypsin insult was chosen as representative of a fecal insult, specifically a fecal protease insult, for the examples that follow.

The insult solution is prepared by diluting a 10 mg/ml stock solution in phosphate-buffered saline to a working concentration of 250 μg/ml. The base of the stock solution is 50 mM NaOAcetate, pH 5.5 and 0.15 M NaCl stored at −80° C. One milliliter of the stock protease insult solution contains 2558 USP units of trypsin and 298 USP units of chymotrypsin and is available from Specialty Enzymes, Inc. of Chino, Calif. The bile acid insult solution can be prepared by dissolving 65 mg of cholic acid, 62 mg of deoxycholic acid and 31 mg of chenodeoxycholic acid in 10 ml of phosphate-buffered saline. The bile acid insult components can also be purchased from Sigma Chemical Co. of St. Louis, Mo. Phosphate-buffered saline, pH 7.4 (hereinafter “PBS”) can be purchased from Life Technologies of Rockville, Md.

After application of the insult solution, the well plates are incubated for six hours in the 37° C., 5% CO₂ incubator. At the end of six hours, the well plates are removed from the incubator, the underlying media is removed and stored at −80° C. The response of the EPIDERM skin culture to the test compositions/control and the insult solution is determined by measuring the amount of interleukin-1 alpha (IL-1α). Interleukin-1 alpha can be quantified using an Interleukin-1 alpha Quantikine Kit available from R&D Systems of Minneapolis, Minn. Interleukin-1 alpha measurements are converted to Log₁₀ for each of the treatments and the averages for each treatment are calculated. In order to determine the ability of the compositions to reduce skin irritation caused by the biological insults, the percent mean reduction of IL-1α is calculated as follows:

$$\% mean reduction of IL - 1 a = 100 \times \frac{\begin{matrix}( ( PJ control + insult ) result - \\ ( test composition + insult ) result )\end{matrix}}{\begin{matrix}( ( PJ control + insult ) result - \\ ( PJ control + PBS ) result )\end{matrix}}$$

(Test composition+insult) result=the measured amount of IL-1α from treatment with a test formulation+insult.

(PJ control+insult) result=the measured amount of IL-1α from a treatment with a control formulation+insult.

(PJ control+PBS) result=the measured amount of IL-1α from a treatment with a control formulation with PBS.

The greater the % mean reduction of IL-1α, the more effective a composition is at reducing irritation caused by the biological insult (proteases or bile acids).

In order to insure that the test compositions/control do not affect the viability of the EPIDERM skin culture, a MTT assay is run. The MTT dye is taken up by the cells. The reduction of the dye as a result of cellular metabolism can be used to measure the cytotoxicity of the test compositions. In order to confirm viability, inserts of the EPIDERM skin culture that have already been subjected to the test compositions and biological insults are removed from their media and are washed consecutively through immersion in three different beakers of PBS. Fresh PBS is used for each test composition or control being evaluated. The PBS is discarded onto paper towel. The EPIDERM skin culture inserts are then patted onto paper towel and placed into the wells of a 24 well plate containing 300 microliters of pre-warmed media. After all of the EPIDERM skin culture inserts are washed, they are transferred to new 24 well plates containing 300 microliters of the MTT reagent. The MTT reagent is thiazolyl blue having the formula 3-[4,5-Dimethylthiazol-2-yl]-2,5-diphenyltetrzoliumbromide. The plates are incubated for 2 hours in a 37° C., 5% CO₂ incubator. After incubation, the EPIDERM skin culture inserts are transferred to 24 well plates and are immersed in 2 milliliters of MTT extraction buffer. The extraction buffer extracts the MTT reagent from the cells. The 24 well plates are parafilmed, covered and placed in ZIPLOCK bags to reduce evaporation of the extraction buffer. The covered plates are rocked overnight in the dark. Following overnight rocking, the liquid in the EPIDERM skin culture inserts is decanted back into the wells. The contents of each well are mixed and a 200 microliter aliquot is then removed from each well and transferred to a 96 well plate. The optical density (OD) of the samples is measured at 570 nm using a spectrophotometer. Five hundred seventy nanometers is the optimal wavelength at which to measure the reduced form of MTT reagent. This reading is subtracted from a background reading at 650 nm to improve data quality. Percent viability of each test composition+insult relative to a negative petrolatum control+PBS is recorded as the Mean OD_{test composition+insult} divided by the Mean OD_{PJ control+PBS}; the quotient then multiplied by 100.

As described above, various experiments were conducted to elucidate the benefits provided by the compositions of the invention. For example, EPIDERM skin culture studies were conducted to measure the reduction in IL-1 α response between compositions of the invention and a fecal protease-induced irritation. The EPIDERM skin culture studies and associated MTT assays were conducted as already described herein and the results are as reported in Table 1. below.

TABLE 1

| | Mean | |
|---|---|---|
| | Reduction of | |
| | Interleukin-1 | |
| | Alpha | Viability |
| Composition | (percentage) | (percentage) |
| 100% Snow White Petrolatum (available | 0%; 32% | 93%; 77% |
| from Penreco) | | |
| 90% Petrolatum; and 10% MQ Resin/1107 | 5%; 77%* | 97%; 84% |
| combination (“1107” is a methyl hydrogen | | |
| polydimethyl siloxane available from Dow | | |
| Corning) | | |
| 90% Petrolatum; and 10% Dow Corning | 64%*; 37%# | 97%; 97% |
| 9506 silicone elastomer powder (available | | |
| from Dow Corning) | | |

*indicates the composition had a significant mean difference from the protease insult applying a Student's t-test with p < 0.05.
#p value = 0.05868

For each of the compositions in Table 1, mean reduction of the inflammatory marker (IL-1 α) for two experiments are shown. For each experiment, five replicates for each composition were evaluated. The IL-1 α reduction results of Table 1 show that the compositions of the invention provide a skin protectant (or barrier enhancing) effect as evidenced by a reduced irritation response.

The reduction of IL-1 α results were analyzed to statistically identify “outlier” results. The EPIDERM skin model is known to be variable with the variability attributed to differences in the culture, variation in the application of treatment and other uncontrollable factors. A statistical analysis technique was applied to identify when a result abnormally deviated from the rest of the data set. The irritation values were first converted to Log₁₀ in order to make them more Gaussian (bell curve-shaped). After conversion, the values were analyzed for high or low value outliers; subsequently, the values were analyzed with a student's t-test to identify significant differences from the “control”. The statistical analysis used to identify “outliers” is described on page 460 of the book, “Statistical Methods in Research and Production” edited by Owen L. Davies and Peter L. Goldsmith, published by Longman Group Limited, fourth revised edition published in 1984.

In addition to providing better barrier properties, the compositions of the invention are believed to be breathable. The breathability of representative compositions of the invention was evaluated using a MOCON water vapor transmission measurement similar to the water vapor transmission rate measurement described herein for the outer cover materials of the diaper 10. However, in order to evaluate the “breathability” of the compositions of the invention, rectangles of collagen film having dimensions of 7.5×10.7 cm were prepared. The target region, a circle with diameter of 5.0 cm, was drawn on the uneven side of the film. The mass of the collagen film rectangle was measured in milligrams, and from that, the initial basis weight (mg/cm²) was determined. The collagen film was then taped, smooth side up, to a piece of weighing paper. This weighing paper was, in turn, taped to the bottom of an incubator heated to ˜30° C.

A film spreader bar was heated to 85° C., while the composition to be tested was heated to 90° C. Once the bar and composition were heated, the spreader bar was placed so that the #1 side (the smallest gap) was next to the collagen film. A target of 1 mg/cm² of composition was poured inside the spreader bar and above the target region. The spreader bar was quickly pulled over the target area to ensure even spreading of the composition. Following application, the composition was allowed to solidify so that the target region could be cut out with a scalpel. The target region was carefully placed composition side up in a weighing boat, and the mass was recorded and the final basis weight (mg/cm²) was determined. Composition add-on levels were calculated by subtracting the initial basis weight from the final basis weight. The results of the MOCON measurements are reported in Table 2. below.

TABLE 2

| Sample Composition (% by weight) | MOCON value (g/m²/24 hr) |
|---|---|
| 88% Petrolatum; 10% Dow Corning 9506 | 169.2 |
| silicone elastomer; 2% silica | |
| 100% Petrolatum | 0.1 |

The results show that the silicone elastomer component provides breathability to the composition.

As already described herein, the compositions of the invention have rheological properties previously unrecognized as being significant to the performance of compositions transferred from absorbent articles to the skin of wearer's of absorbent articles. The properties of Tangent Delta, softening temperature, elastic modulus and the temperature slope of the elastic modulus curve have been found to be relevant to the ability of the composition to distribute or spread evenly when transferred to the skin and to form a stable film. These properties have been determined for the compositions of the invention and for previously known compositions in order to demonstrate the improvement provided by the compositions of the invention.

Tangent Delta Measurement Procedure

The Dynamic Mechanical Analysis (“DMA”, includes elastic modulus, softening temperature and slope measurements) were run on a RSA II Solids Analyzer available from Rheometric Scientific, Piscataway, N.J. The tests were executed in shear mode, by using a “shear sandwich” attachment, having a sample length of 15 mm, a sample width of 15 mm and a sample thickness of 1 mm. To determine the softening temperature (T_{soft}) and the slope of the elastic modulus (G′) at temperatures higher than T_{soft}, the tests were run under constant strain amplitude oscillations with a frequency of 2 Hz and amplitude 2%. Each DMA test was performed with step intervals over the temperature range from −60° C. to +70° C., with a step of 2° C. and dwell time of 2 seconds per point. The instrument records the temperature changes of the loss modulus (E″), the elastic modulus (E′) and their ratio (Tangent Delta) as a function of the temperature. In order to determine Tangent Delta at low deformation rates, the samples were tested at two predefined temperatures (35° C. and 40° C.) under constant strain amplitude of 1% and frequency of 0.1 Hz. The shear sandwich attachment is the same as that used for the DMA tests.

The DMA tests and Tangent Delta Measurement were made for the compositions identified in Table 3. below. The Tangent Delta, softening temperature and temperature slope are reported for each composition.

TABLE 3

| | Tangent | | |
|---|---|---|---|
| | Delta | | |
| | (35° C.- | | |
| | 40° C.) | Soft- | |
| | Fre- | ening | |
| | quency/ | Temp- | |
| | Amplitude | erature | |
| Sample Descriptions | (0.1 Hertz/ | (deg. | |
| (% by weight) | 1%) | C.) | Slope |
| 50% White Petrolatum (White Protopet 1S | 1.05* | +19 | −0.07 |
| obtained from Witco-Crompton) | | | |
| 35% Cetearyl Alcohol (obtained from Glenn | | | |
| Corp.) | | | |
| 15% Steareth-2 (obtained from ICI Specialty | | | |
| Chemicals) | | | |
| (described as Example 5 at Col. 22, lines | | | |
| 1-24 of U.S. Pat. No. 5,643,588 issued | | | |
| Jul. 1, 1997 to Roe et al.) | | | |
| 60% Snow White Petrolatum (available from | 1.2 | +27 | −0.08 |
| Penreco) | | | |
| 34% Wax Blend (ozokerite wax available | | | |
| from International Group, Inc.) | | | |
| 6% ELVAX 410 resin (available from E.I. | | | |
| DuPont) | | | |
| (ointment provided on HUGGIES diapers | | | |
| sold by Kimberly-Clark Corporation) | | | |
| 77.7% Ultrapure L Petrolatum (available | 0.78 | −18 | −0.08 |
| from Ultra Chemical Company) | | | |
| 5% Wax Blend (ozokerite wax available | | | |
| from International Group, Inc.) | | | |
| 10% Sunflower Oil (available from R.I.T.A. | | | |
| Corp.) | | | |
| 1% PROLIPID 141 blend (available from | | | |
| International Specialty Products) | | | |
| 0.8% Soy Sterol (available from Henkel | | | |
| Corp.) | | | |
| 5% Glycerin (available from Glenn Corp.) | | | |
| 0.5% Sorbitan monooleate (available from | | | |
| ICI Specialty Chemicals) | | | |
| COMPOSITION OF THE INVENTION: | 0.45 | +20 | −0.06 |
| 53% Snow White Petrolatum (available from | | | |
| Penreco) | | | |
| 35% Wax Blend (ozokerite wax available | | | |
| from International Group, Inc.) | | | |
| 10% Dow Corning 9506 silicone elastomer | | | |
| powder (available from Dow Corning) | | | |
| 2% Cab-O-Sil M5P (available from Cabot | | | |
| Corp.) | | | |
| 40% Snow White Petrolatum (available from | 0.05* | +36 | −0.09 |
| Penreco) | | | |
| 60% Carnuba Wax (available from Koster | | | |
| Keunen Inc.) | | | |
| 100% Snow White Petrolatum (available | 1.05 | −18 | −0.06 |
| from Penreco) | | | |
| 70% Snow White Petrolatum (available from | 0.74 | +18 | −0.07 |
| Penreco) | | | |
| 30% Wax Blend (ozokerite wax available | | | |
| from International Group, Inc.) | | | |
| *These two samples had to be prepared at a temperature greater than room temperature (about 50° C.) in order to be applied to the shear sandwich attachment (the other samples were soft enough to be applied at room temperature). As a result, these samples may have experienced a phase change between 35 and 40° C. and therefore, it is not clear if the material was completely homogenous when the measurements were taken. The Tangent Delta values were not stable. | | | |
| Penreco is located in Karns City, Pennsylvania; Witco-Crompton Corp. is located in Petrolia, Pennsylvania; E.I. DuPont is located in Wilmington, Delaware; Glenn Corp. is located in St. Paul, Minnesota; ICI Specialty Chemicals is located in Wilmington, Delaware; International Group, Inc. is located in Wayne, Pennsylvania; Ultra Chemical Company is located in Red Bank, New Jersey; R.I.T.A. Corp. is located in Woodstock, Illinois; International Specialty Products located in | | | |
| # Parsippany, New Jersey; Henkel Corporation is located in Ambler, Pennsylvania; Dow Corning is located in Midland, Michigan; Cabot Corp. is located in Tuscola, Illinois; and Koster Kuenen is located in Watertown, Connecticut. | | | |

The results of Table 3. demonstrate that the Tangent Delta values for previously known compositions are substantially greater than 0.65. The higher Tangent Delta values are representative of relatively greater loss moduli (G″) than elastic moduli (G′), meaning that the composition spreads well but does not form a stable film. The composition representative of the invention has a Tangent Delta value in the desired range (0.45) indicating the desired balance between spreadability and stable film-forming properties. The petrolatum/carnuba wax has a very low Tangent Delta value, which suggests poor spreadability but good stability. The “softening temperature” is that temperature at which at least one phase of the composition starts to melt. The temperature slope is the rate at which the elastic modulus (G′) drops as temperature is increased. The method by which the softening temperature and the temperature slope are determined is shown in FIG. 2. FIG. 2 represents a plot of elastic modulus versus temperature for a composition of the invention. The softening temperature is the temperature at which the slope of the elastic modulus curve changes and the temperature slope is the slope of the elastic modulus curve at temperatures greater than the softening temperature.

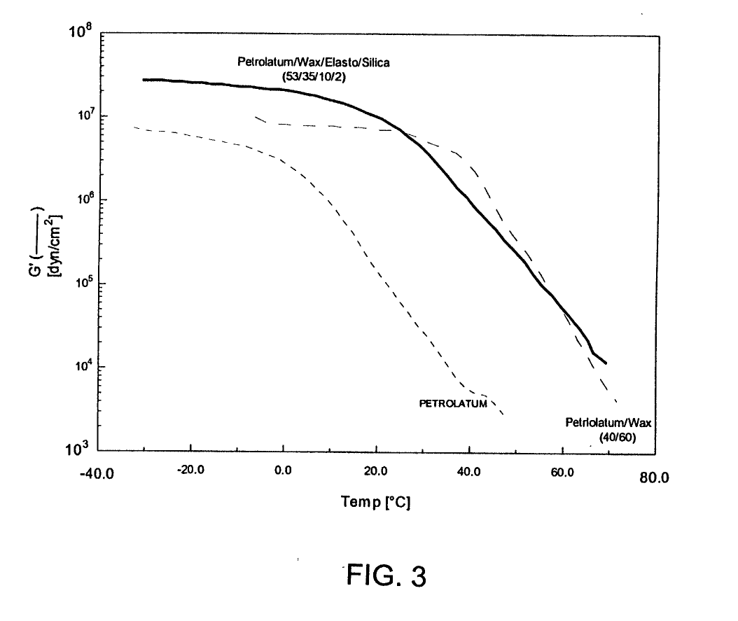

FIG. 3 graphically represents the elastic modulus (G′) versus temperature for three compositions: composition of the invention; blend of petrolatum and carnauba wax; and pure petrolatum. A high elastic modulus corresponds to a hard material and a low elastic modulus corresponds to a softer material. The pure petrolatum has the lowest softening temperature and the petrolatum/carnuba wax blend has the highest softening temperature. The composition of the invention has the lowest temperature slope.

The preceding test results representatively illustrate that the compositions of the present invention improve the barrier properties of the skin while still being breathable so as to not occlude the skin's natural respiration. Further, the compositions of the invention have improved Theological properties that deliver a desirable balance of distribution and spreading ability and the ability to form a stable film. The benefits of the compositions of the invention are desirably achieved through use of the compositions on absorbent articles such as disposable diapers.

While the invention has been described in detail with respect to the specific aspects thereof, it will be appreciated that those skilled in the art, upon attaining an understanding of the foregoing, may readily conceive of alterations to, variations of, and equivalents to these aspects. Accordingly, the scope of the present invention should be assessed as that of the appended claims and any equivalents thereto.

We claim:

1. An absorbent article comprising:
(a) an outer cover;
(b) a liquid permeable bodyside liner that defines a bodyfacing surface and that is connected in superposed relation to the outer cover;
(c) an absorbent body that is located between the bodyside liner and the outer cover; and
(d) a composition on at least a portion of the bodyfacing surface of the bodyside liner wherein the composition has a Tangent Delta value of from about 0.10 to about 0.65 measured over a temperature range of 35 to 40 degrees Celsius according to a Tangent Delta Measurement Procedure set forth herein.

2. The absorbent article of claim 1 wherein the composition has a softening temperature of about 15 deg. C to about 30 deg. C.

3. The absorbent article of claim 2 wherein the composition has an elastic modulus of from about 10⁵ dynes/cm² to about 10⁷ dynes/cm².

4. The absorbent article of claim 3 wherein the elastic modulus has a temperature slope of from about −0.06 to about −0.08.

5. The absorbent article of claim 1, wherein the composition has a Tangent Delta value of from about 0.35 to about 0.55 measured over a temperature range of 35 to 40 degrees Celsius according to a Tangent Delta Measurement Procedure set forth herein.

6. The absorbent article of claim 1, wherein the composition includes from about 40 to about 95 percent by weight of emollient, from about 0.1 to about 40 percent by weight of viscosity enhancer and from about 0.1 to about 20 percent by weight of silicone elastomer.

7. An absorbent article comprising:
(a) an outer cover;
(b) a liquid permeable bodyside liner that defines a bodyfacing surface and that is connected in superposed relation to the outer cover;
(c) an absorbent body that is located between the bodyside liner and the outer cover; and
(d) a composition on at least a portion of the bodyfacing surface of the bodyside liner that includes from about 40 to about 95 percent by weight of emollient, from about 0.1 to about 40 percent by weight of viscosity enhancer and from about 0.1 to about 20 percent by weight of silicone elastomer.

8. The absorbent article of claim 7, wherein the emollient of the composition is selected from petrolatum, vegetable based oils, mineral oils, dimethicone, lanolin, glycerol esters, alkoxylated carboxylic acids, alkoxylated alcohols, fatty alcohols and mixtures thereof.

9. The absorbent article of claim 7, wherein the viscosity enhancer of the composition is selected from polyolefin resins, lipophilic/oil thickeners, ethylene/vinyl acetate copolymers, quaternary starch compounds, natural clays, synthetic analogs of natural clays, organically modified clays, quaternary modified clays, polyethylene, silica, silica silylate, silica methyl silylate, colloidal silicone dioxide, alkyl hydroxy ethyl cellulose, microcrystalline wax, shellac wax, hexadecyl cosanyl hexacosanate, C₂₀-C₄₀ alkyl hydroxystearyl stearate, glycol montanate, ozokerite wax, polyperfluoromethylisopropylether montan wax and mixtures thereof.

10. The absorbent article of claim 7, wherein the silicone elastomer of the composition is selected from crosslinked non-emulsifying siloxane elastomers formed from a divinyl compound reacted with Si—H linkages of a polysiloxane; crosslinked non-emulsifying siloxane elastomers formed from a C₃-C₂₀ alkyl polysiloxane compound reacted with Si—H linkages of a polysiloxane; and mixtures thereof.

11. The absorbent article of claim 7, wherein the silicone elastomer of the composition is selected from vinyl MQ resin/organopolysiloxane crosspolymers in which the organo group is selected from polyglycol, polyglycerol, oligosaccharide, hydroxyl-terminated polyoxyalkylene polyethers, carboxylate esters of hydroxyl-terminated polyoxyalkylene polyethers, lower alkanol ethers and mixtures thereof.

12. The absorbent article of claim 10, wherein the crosslinked non-emulsifying siloxane elastomers are selected from Vinyl Dimethicone/Methicone Crosspolymer, Crosslinked Stearyl Methyl Dimethyl Siloxane Copolymer, Dimethicone/Vinyl Dimethicone Crosspolymer, Dimethicone/Phenyl Vinyl Dimethicone Crosspolymer and mixtures thereof.

13. The absorbent article of claim 7 wherein the composition further includes from about 5 to about 59 percent by weight of solidifying agent.

14. The absorbent article of claim 13, wherein the solidifying agent is selected from beeswax, behenyl behenate, behenyl benzoate, branched esters, candelilla wax, carnauba wax, synthetic carnauba wax, PEG-12 carnauba wax, cerasin, microcrystalline wax, hydrogenated microcrystalline wax, hexadecylcosanyl hexacosanate, polyperfluoromethylisopropylether montan wax, alkylmethylsiloxanes, glycol montanate, jojoba wax, lanolin wax, ozokerite, paraffin, synthetic paraffin, polyethylene, C₂₀-C₄₀ alkyl hydroxystearyl stearate, C₃₀ alkyl dimethicone, cetyl esters, zinc stearate, shellac wax, hydrogenated cottonseed oil, hydrogenated squalene, hydrogenated jojoba oil and mixtures thereof.

15. The absorbent article of claim 7 wherein the composition further includes from about 0.1 to about 59 percent by weight of natural fats or oils.

16. The absorbent article of claim 15, wherein the natural fat or oil is selected from Avocado Oil, Apricot Oil, Babassu Oil, Borage Oil, Camellia Oil, Canola Oil, Castor Oil, Coconut Oil, Corn Oil, Cottonseed Oil, Evening Primrose Oil, Hydrogenated Cottonseed Oil, Hydrogenated Palm Kernel Oil, Maleated Soybean Oil, Meadowfoam Oil, Palm Kernel Oil, Peanut Oil, Rapeseed Oil, Safflower Oil, Sphingolipids, Sweet Almond Oil, Tall Oil, Lauric Acid, Palmitic Acid, Stearic Acid, Linoleic Acid, Stearyl Alcohol, Lauryl Alcohol, Myristyl Alcohol, Behenyl Alcohol, Rose Hip Oil, Calendula Oil, Chamomile Oil, Eucalyptus Oil, Juniper Oil, Sandlewood Oil, Tea Tree Oil, Sunflower Oil, Soybean Oil and mixtures thereof.

17. The absorbent article of claim 7 wherein the composition further includes from about 0.1 to about 10 percent by weight of sterols or sterol derivatives.

18. The absorbent article of claim 17, wherein the sterol or sterol derivative is selected from cholesterol, sitosterol, stigmasterol, and ergosterol, as well as, C₁₀-C₃₀ cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyldecanoate, dihydrolanosterol, dihydrolanosteryl octyldecanoate, ergocalciferol, tall oil sterol, soy sterol acetate, lanasterol, soy sterol, avocado sterols, sterol esters and mixtures thereof.

19. The absorbent article of claim 7 wherein the composition further includes a skin care ingredient selected from allantoin and its derivatives, aloe, aluminum hydroxide gel, calamine, cocoa butter, cod liver oil, dimethicone, glycerin, kaolin and its derivatives, lanolin and its derivatives, mineral oil, petrolatum, white petrolatum, shark liver oil, talc, topical starch, zinc acetate, zinc carbonate, zinc oxide and mixtures thereof.

20. The absorbent article of claim 7 wherein the composition has a Tangent Delta value of from about 0.10 to about 0.65 measured over a temperature range of 35 to 40 degrees Celsius according to a Tangent Delta Measurement Procedure set forth herein.

21. The absorbent article of claim 7 wherein the composition has a softening temperature of 15 deg. C to about 30 deg. C.

22. The absorbent article of claim 7 wherein the composition has an elastic modulus of from about 10⁵ dynes/cm² to about 10⁷ dynes/cm².

23. The absorbent article of claim 7 wherein the elastic modulus has a temperature slope of from about −0.06 to about −0.08.
